(19) United States
(12) Patent Application Publication
(10) Pub. No.: US 2019/0183463 A1
(43) Pub. Date: Jun. 20, 2019

(54) TISSUE COLLECTION AND PROCESSING CASSETTE WITH APPLIED IMAGING

(71) Applicant: Devicor Medical Products, Inc., Cincinnati, OH (US)

(72) Inventor: Bryan R. Keller, Loveland, OH (US)

(21) Appl. No.: 16/223,468

(22) Filed: Dec. 18, 2018

Related U.S. Application Data
(60) Provisional application No. 62/607,765, filed on Dec. 19, 2017.

(51) Int. Cl. A61B 10/00; A61B 10/02; G01N 23/04

(52) U.S. Cl. CPC A61B 10/0096; A61B 10/0275; G01N 2223/6126; A61B 2010/0225; G01N 23/04

(57) ABSTRACT

A biopsy device includes a body, a needle, a tissue sample holder, a tissue processing cassette, and an x-ray sensor. The needle extends distally from the body and is in communication with the tissue sample holder. The tissue sample holder includes a rotatable member defining a cassette mount and a sensor mount proximate to the cassette mount. The tissue processing cassette is sized to be used in a pathology laboratory for purposes of dehydrating, embedding and sectioning. The tissue processing cassette is configured for receipt within the cassette mount of the tissue sample holder for receipt of one or more tissue samples. The x-ray sensor is configured to be received within the sensor mount and is adapted to receive an x-ray that has passed through one or more tissue samples received in the tissue processing cassette.

### PRIORITY

This application claims priority to U.S. Provisional Patent App. No. 62/607,765 entitled “Tissue Collection and Processing Cassette with Applied Imaging,” filed Dec. 19, 2017, the disclosure of which is incorporated by reference herein.

### BACKGROUND

A biopsy is the removal of a tissue sample to examine tissue for signs of cancer or other disorders. Tissue samples are obtained in a variety of ways using various medical procedures involving a variety of the sample collection devices. For example, biopsies may be open (surgically removing tissue) or percutaneous (e.g. by fine needle aspiration, core needle biopsy or vacuum assisted biopsy). After the tissue sample is collected, the tissue sample is analyzed at a lab (e.g. a pathology lab, biomedical lab, etc.) that is set up to perform the appropriate tests (such as histological analysis).

Biopsy samples have been obtained in a variety of ways in various medical procedures including open and percutaneous methods using a variety of devices. For instance, some biopsy devices may be fully operable by a user using a single hand, and with a single insertion, to capture one or more biopsy samples from a patient. In addition, some biopsy devices may be tethered to a vacuum module and/or control module, such as for communication of fluids (e.g., pressurized air, saline, atmospheric air, vacuum, etc.), for communication of power, and/or for communication of commands and the like. Other biopsy devices may be fully or at least partially operable without being tethered or otherwise connected with another device. Biopsy devices may be used under stereotactic guidance, ultrasound guidance, MRI guidance, Positron Emission Mammography (“PEM” guidance), Breast-Specific Gamma Imaging (“BSGI”) guidance or otherwise.

At several steps during tissue processing using conventional techniques and instruments, it may be necessary to manually manipulate the tissue. This manual manipulation takes time and introduces the possibility of human error causing mistakes during the processing of tissue. Any human error during the processing of tissue can make the pathological examination of the tissue much more problematic to achieve the desired goal of having an accurate diagnosis. Thus, it is understood that a desired goal of modern tissue processing is the reduction of the requirement that tissue be manually manipulated.

Various devices and techniques for tissue handling are disclosed in International Pat. Pub. No. WO 2013/192606, entitled “Biopsy Tissue Sample Transport Device and Method of Using Thereof,” published on Dec. 27, 2013; International Pat. Pub. No. WO 2013/192607, entitled “Tissue Sample Container and Methods,” published on Dec. 27, 2013; International Pat. Pub. No. WO 2014/151603, entitled “Biopsy Device,” published on Sep. 25, 2014; U.S. Pat. No. 7,715,523, entitled “System and Apparatus for Rapid Stereotactic Breast Biopsy Analysis,” issued on May 11, 2010; U.S. Pat. No. 8,503,602, entitled “System and Apparatus for Rapid Stereotactic Breast Biopsy Analysis,” issued on Aug. 6, 2013; U.S. Pat. No. 8,485,987, entitled “Tissue Handling System with Reduced Operator Exposure,” issued Jul. 16, 2016; U.S. Pat. No. 8,802,034, “Tissue Container for Molecular and Histology Diagnostics Incorporating a Breakable Membrane,” issued on Aug. 12, 2014; and U.S. Pat. No. 9,056,317, “Tissue Container for Molecular and Histology Diagnostics Incorporating a Breakable Membrane,” issued on Jun. 16, 2016. The disclosure of each of the above-cited U.S. Patents is incorporated by reference herein.

While several systems and methods have been made and used for obtaining and processing a biopsy sample, it is believed that no one prior to the inventor has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements. In the drawings some components or portions of components are shown in phantom as depicted by broken lines.

FIG. 1 depicts a perspective view of an exemplary biopsy device;

FIG. 2 depicts an exploded perspective view of a tissue sample holder assembly of the biopsy device of FIG. 1;

FIG. 3 depicts a perspective view of a tissue sample tray of the tissue sample holder assembly of FIG. 2, with the tissue sample tray in an arcuate configuration;

FIG. 4 depicts a perspective view of the tissue sample tray of FIG. 3 in a flattened configuration;

FIG. 5 depicts a front elevational view of the tissue sample tray of FIG. 3 disposed within ajar;

FIG. 6 depicts a perspective view of an exemplary sample cassette for use in processing tissue sample collected with the biopsy device of FIG. 1;

FIG. 7 depicts a flowchart of an exemplary tissue collection and analysis work flow for use with the biopsy device of FIG. 1 and the sample cassette of FIG. 6;

FIG. 8 depicts a perspective view of an exemplary cassette assembly that may be readily used with the biopsy device of FIG. 1 in lieu of the tissue sample tray of FIG. 3 and/or the sample cassette of FIG. 6;

FIG. 9 depict another perspective view of the cassette assembly of FIG. 8;

FIG. 10 depicts an exploded perspective view of the cassette assembly of FIG. 8;

FIG. 11 depicts a perspective view of an exemplary cassette tray of the cassette assembly of FIG. 8;

FIG. 12 depicts another perspective view of the cassette tray of FIG. 11;

FIG. 13 depicts a perspective view of an exemplary cover of the cassette assembly of FIG. 8;

FIG. 14 depicts another perspective view of the cover of FIG. 13;

FIG. 15A depicts a side cross-sectional view of the cassette assembly of FIG. 8, with the cassette tray of FIG. 11 initially inserted into the cover of FIG. 13;

FIG. 15B depicts another side cross-sectional view of the cassette assembly of FIG. 8, with the cassette tray of FIG. 11 intermediately inserted into the cover of FIG. 13;

FIG. 15C depicts still another side cross-sectional view of the cassette assembly of FIG. 8, with the cassette tray of FIG. 11 fully inserted into the cover of FIG. 13;

FIG. 16 depicts a perspective view of an exemplary alternative tissue sample holder assembly, including the cassette tray of FIG. 8 received in a cassette manifold and an exemplary sensor received in a sensor manifold;

FIG. 17 depicts a perspective view of the tissue sample holder assembly of FIG. 16, excluding the cassette tray of FIG. 8 and the sensor of FIG. 16;

FIG. 18 depicts a perspective view of the tissue sample holder assembly of FIG. 16 including a plurality of access and vacuum openings;

FIG. 19 depicts a front elevational view of the tissue sample holder assembly of

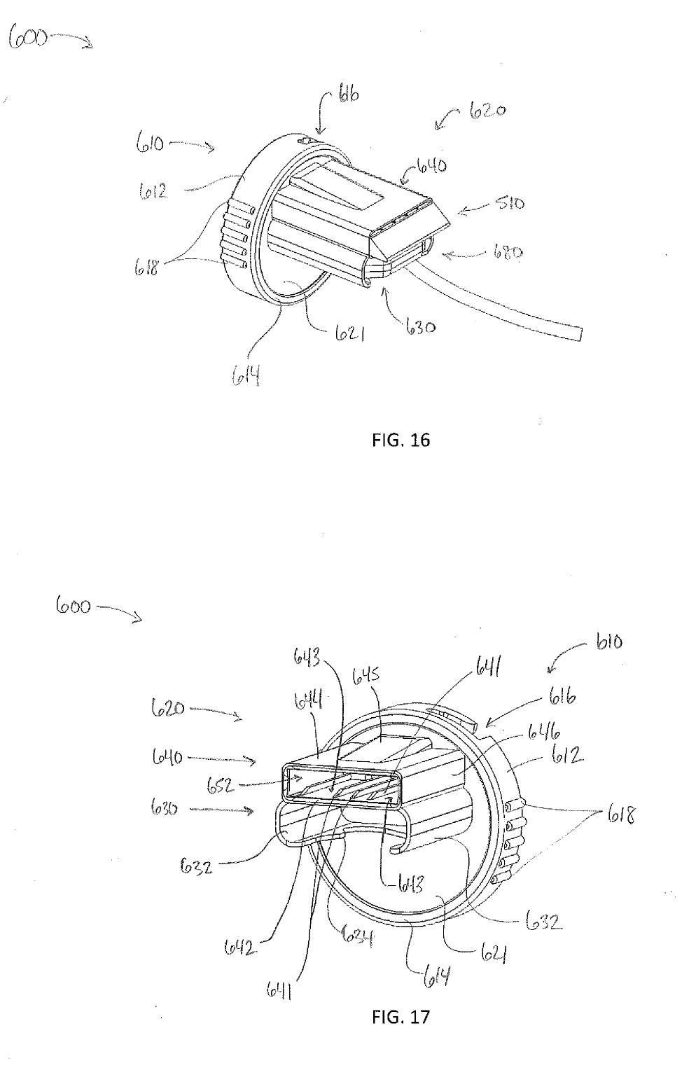

FIG. 16 with the plurality of access and vacuum openings extending through the cassette manifold;

FIG. 20 depicts a perspective view of the tissue sample holder assembly of FIG. 16 with the sensor manifold including a flexible detent for securing the sensor therein;

FIG. 21 depicts a bottom plan view of the tissue sample holder assembly of FIG. 16 with the flexible detent securing the sensor in the sensor manifold;

FIG. 22 depicts a perspective view of the sensor of FIG. 16;

FIG. 23 depicts an exploded perspective view of the sensor of FIG. 16;

FIG. 24 depicts a perspective view of an exemplary imaging device;

FIG. 25 depicts a schematic diagram of the imaging device of FIG. 24 transmitting a beam towards the sensor of FIG. 16, with the sensor outputting a processed image of the contents contained in the tissue sample holder assembly; and

FIG. 26A depicts a side elevational view of the tissue sample holder assembly of

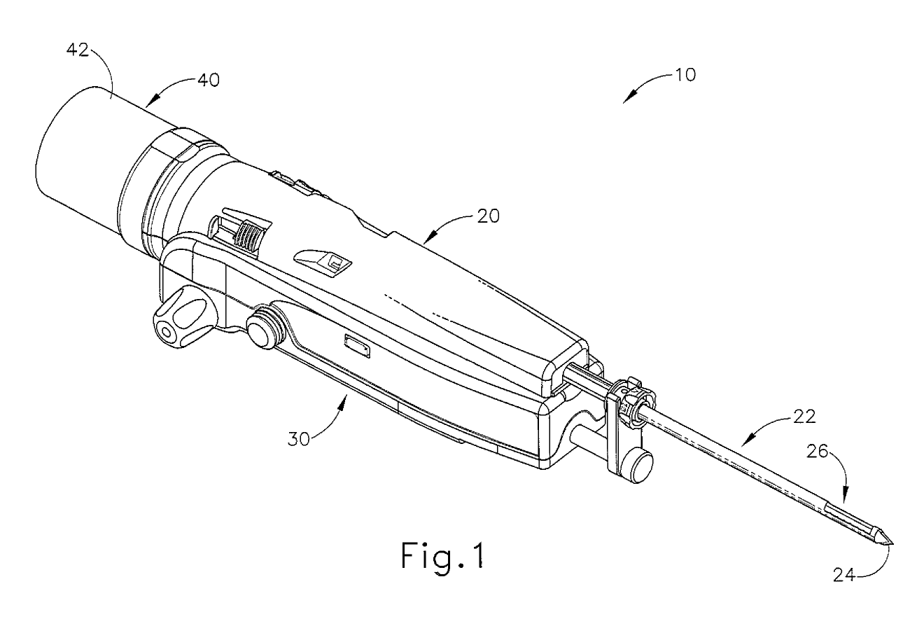

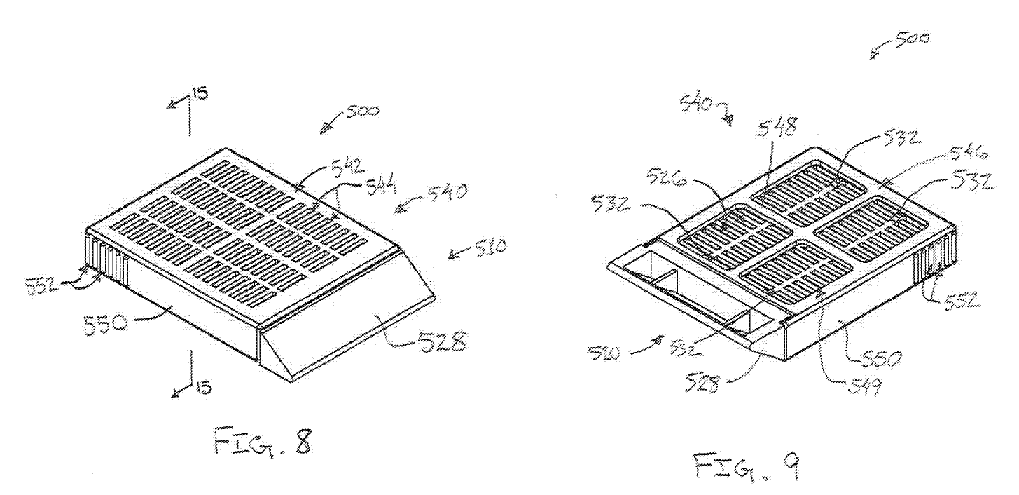

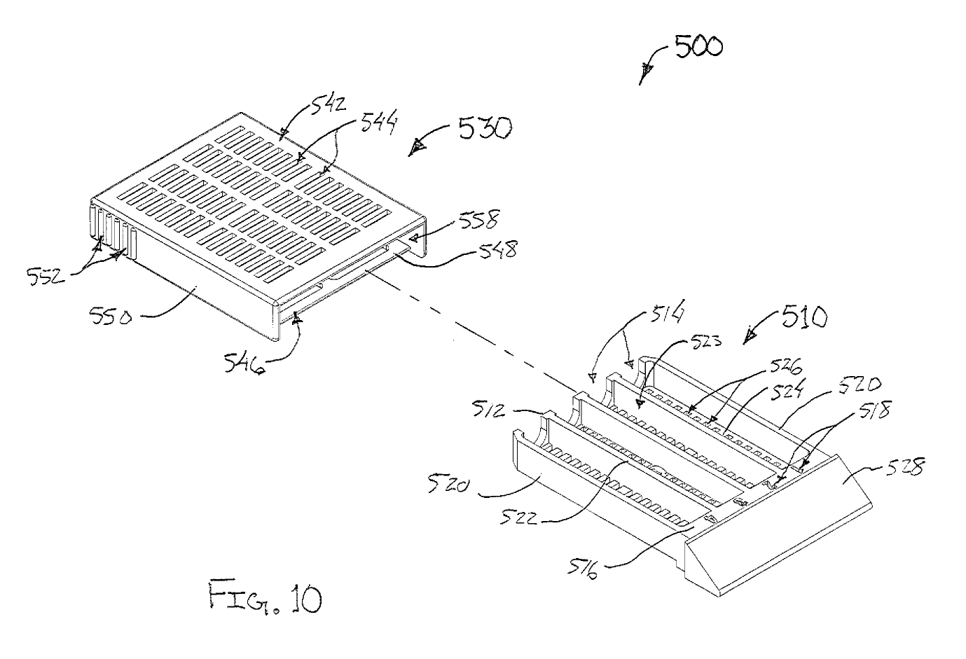

FIG. 16 assembled to the biopsy device of FIG. 1; with the cassette tray of FIG. 8 advanced toward the cassette manifold;

FIG. 26B depicts a side elevational view of the tissue sample holder assembly of FIG. 16 assembled to the biopsy device of FIG. 1; with the sensor of FIG. 16 advanced toward the sensor manifold;

FIG. 27 depicts the imaging device of FIG. 24 positioned adjacent to the assembled combination of the biopsy device of FIG. 1 and the tissue sample holder assembly of FIG. 16;

FIG. 28A depicts the tissue sample holder assembly of FIG. 16 with the cassette tray of FIG. 8 and the sensor of FIG. 16 received therein, the tissue sample holder assembly rotated to an alternative rotational position;

FIG. 28B depicts the tissue sample holder assembly of FIG. 16 with the cassette tray of FIG. 8 and the sensor of FIG. 16 received therein, the tissue sample holder assembly rotated to another alternative rotational position; and

FIG. 29 depicts a cross sectional view of the assembly of FIG. 27, with tissue samples extracted from the biopsy device of FIG. 1 deposited within the sample tray of FIG. 8, the cross section taken along line 29-29 of FIG. 28A.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

I. Exemplary Biopsy Device

FIG. 1 depicts an exemplary biopsy device (10) that can be used to acquire tissue samples from a patient. Biopsy device (10) comprises a probe assembly (20), a holster assembly (30), and a tissue sample holder assembly (40). Probe assembly (20) includes a distally projecting needle (22) that has a tissue piercing tip (24) and a lateral aperture (26) that is located proximal to tip (24). A tubular cutter (not shown) is slidably disposed in needle (22) and is operable to sever tissue that is protruding through lateral aperture (26). The severed tissue samples are communicated proximally through the lumen of the cutter to tissue sample holder assembly (40), as described below. In some versions, probe assembly (20) is coupled with a control module that is operable to provide communication of vacuum, saline, and/or atmospheric air to probe assembly (20).

Holster assembly (30) includes features that are operable to drive the cutter, features that are operable to fire needle (22) distally into tissue, and features that are operable to rotate needle (22) about a longitudinal axis of needle (22). In some versions, holster assembly (30) is coupled with a control module via a cable that is operable to provide electrical power and/or other electrical signals to holster assembly (30). In addition, or in the alternative, holster assembly (30) may receive a pressurized medium (e.g., air, hydraulic fluid, etc.) in order to provide motive force to drive the cutter of probe assembly (20).

In the present example, probe assembly (20) and holster assembly (30) are configured for use in a stereotactic image guided biopsy procedure. By way of example only, probe assembly (20) and holster assembly (30) may be constructed and operable in accordance with at least some of the teachings of U.S. Pub. No. 2014/0039343, entitled “Biopsy System,” published Feb. 6, 2014, the disclosure of which is incorporated by reference herein. Alternatively, probe assembly (20) and holster assembly (30) may be configured for use in (or otherwise be used in) an ultrasound image guided biopsy procedure and/or an MRI guided biopsy procedure. By way of further example only, probe assembly (20) and holster assembly (30) may be constructed and operable in accordance with at least some of the teachings of U.S. Pub. No. 2013/0150751, entitled “Biopsy Device with Slide-In Probe,” published Jun. 13, 2013, the disclosure of which is incorporated by reference herein. Alternatively, probe assembly (20) and holster assembly (30) may be constructed and operable in any other suitable fashion.

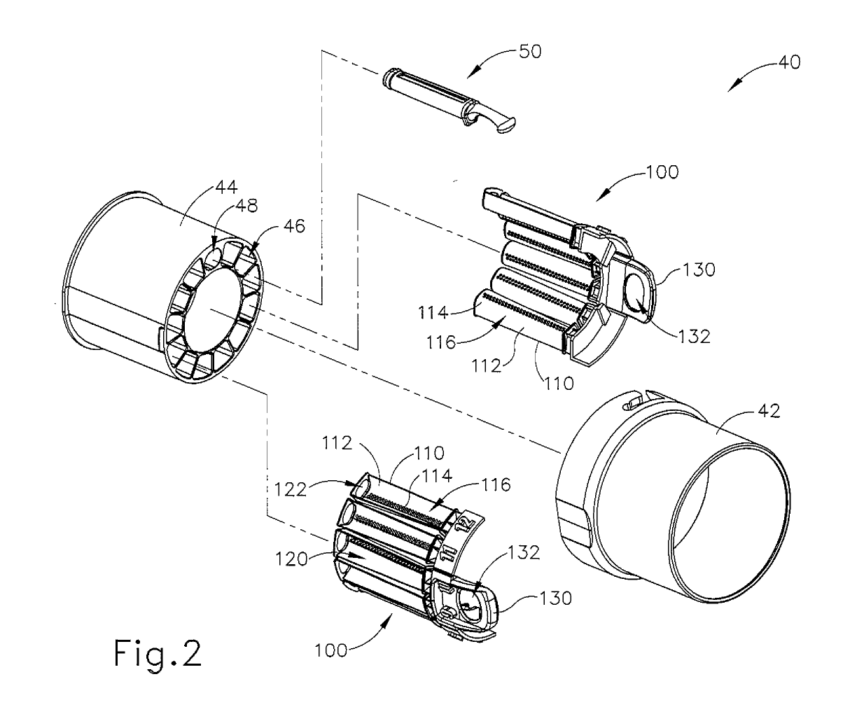

As noted above, tissue sample holder assembly (40) is configured to receive tissue samples that are severed by the cutter from tissue protruding through lateral aperture (26). As shown in FIG. 2, tissue sample holder assembly (40) of this example comprises a cylindraceous outer cover (42) that is removably coupled with probe assembly (20). A rotatable (44) member is rotatably positioned within cover (42). Rotatable member (44) defines an angularly spaced array of strip receiving chambers (46) and a plug chamber (48), such that chambers (46, 48) together an annular arrangement. Rotatable member (44) is rotatable relative to probe assembly (20) to selectively index chambers (46, 48) relative to the cutter. In some versions, drive components in holster assembly (30) drive rotation of rotatable member (44). In some other versions, rotatable member (44) is driven manually by the operator manually grasping some portion of tissue sample holder assembly (40).

As also shown in FIG. 2, tissue sample holder assembly (40) further includes a pair of tissue sample trays (100). Each tissue sample tray (100) comprises a set of distally projecting tissue sample strips (110). Each tissue sample strip (110) is configured for removable insertion into a corresponding strip receiving chamber (46) of rotatable member (44). Each tissue sample strip (110) comprises a set of strip sidewalls (112) joined by a floor (114). Strip sidewalls (112) and floor (114) cooperate to define a tissue receiving chamber (120), such that each tissue sample strip (110) is configured to receive a corresponding tissue sample. Floor (114) defines a plurality of openings (116) that are sized to provide communication of suction and fluids therethrough, while preventing communication of tissue samples therethrough. It should be understood that suction may be communicated through strip receiving chambers (46) to reach tissue receiving chambers (120) via openings (116). Each tissue sample strip (110) of the present example also includes a distal opening (122). Distal opening (122) is sized and configured to enable a severed tissue sample to pass therethrough in order for the tissue sample to be deposited into tissue receiving chamber (120).

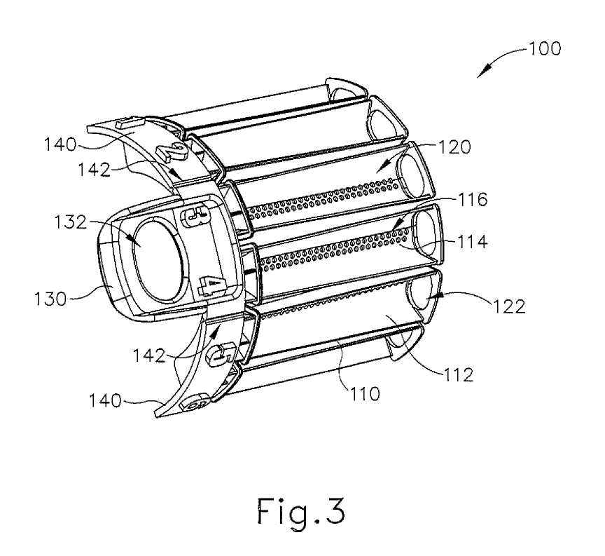

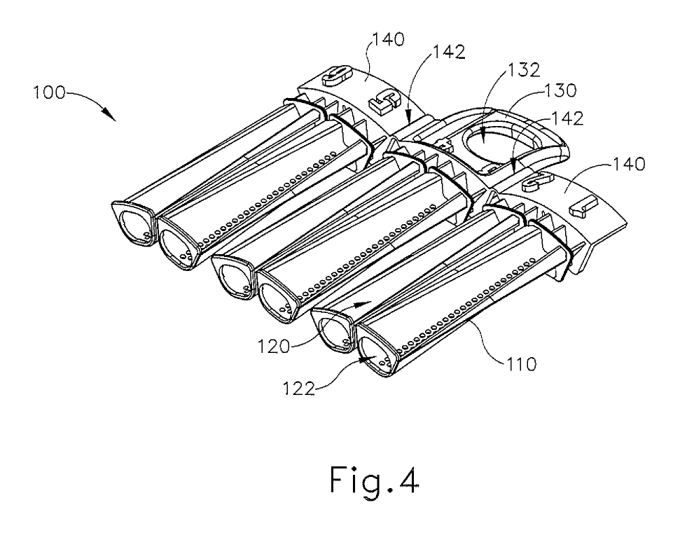

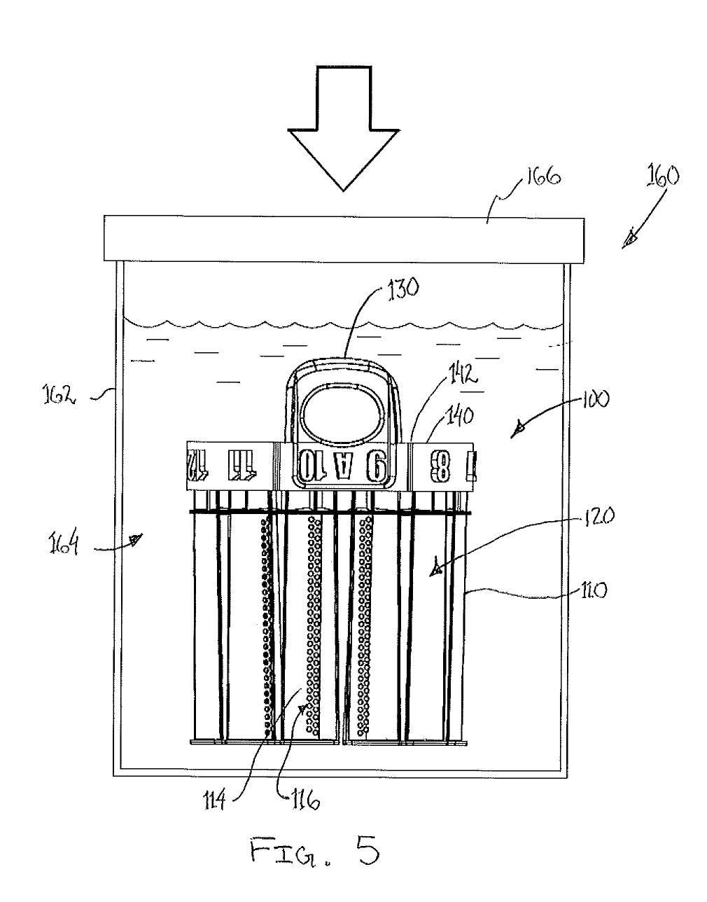

As best seen in FIGS. 3-4, each tissue sample tray (100) further includes a proximally projecting pull tab (130) that defines a tab opening (132). Pull tab (130) is configured to facilitate grasping of tissue sample tray (100) by an operator. Tissue sample tray (100) also includes a set of proximal panels (140). In the present example, two tissue sample strips (110) project distally relative to a corresponding panel (140) of the set of panels (140). Pull tab (130) projects proximally from the centrally positioned panel (140). Panels (140) are flexibly joined together by living hinges (142). Living hinges (142) enable tissue sample tray (100) to transition between the arcuate configuration shown in FIG. 3 and the flattened configuration shown in FIG. 4. In the arcuate configuration, tissue sample tray (100) is configured to fit in rotatable member (44). In the flattened configuration, tissue sample tray (100) is configured to fit in a container (200) as will be described in greater detail below.

As noted above, rotatable member (44) is rotatable relative to probe assembly (20) to selectively index strip receiving chambers (46) relative to the cutter, to thereby selectively index tissue receiving chambers (120) of tissue sample strips (110) relative to the cutter. Rotatable member (44) is also operable to index plug receiving chamber (48) relative to the cutter. When rotatable member (44) is angularly positioned to index plug receiving chamber (48) relative to the cutter, plug (50) may be removed from plug receiving chamber (48) to enable insertion of a biopsy site marker applier instrument (or some other kind of instrument) through the cutter and needle assembly (22), thereby providing an access path to the biopsy site via lateral aperture (26). Otherwise, plug (50) may be left in plug receiving chamber (48) during operation of biopsy device (10), thereby sealing plug receiving chamber (48).

By way of example only, tissue sample holder (40) may be configured and operable in accordance with at least some of the teachings of U.S. Pub. No. 2014/0039343, entitled “Biopsy System,” published Feb. 6, 2014, the disclosure of which is incorporated by reference herein and/or U.S. Pub. No. 2014/0275999, entitled “Biopsy Device,” published Sep. 18, 2014, the disclosure of which is incorporated by reference herein.

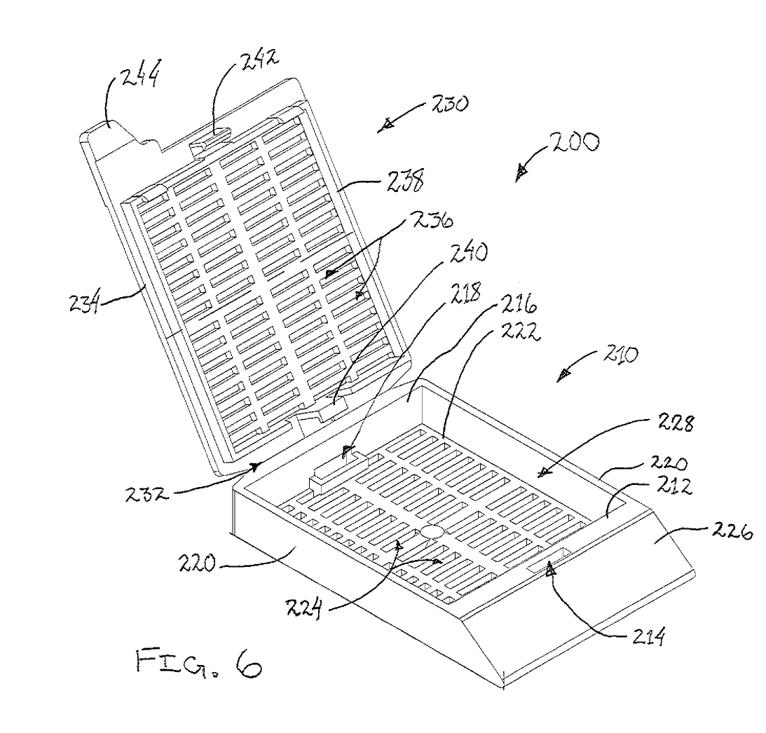

In some instances it may be desirable to insert tissue sample tray (100) in a preservative or other protective medium after collecting tissue samples within each tissue receiving chamber (120) of tissue sample tray (100). As seen in FIG. 5, in some examples tissue sample tray (100) may be used in connection with jar (160). Jar (160) is generally configured to receive one or more tissue sample trays (100) after collection of tissue samples using biopsy device (10). As will be described in greater detail below, jar (160) may be used to transport or store tissue samples once one or more tissue sample trays (100) are deposited therein.

In the present example, jar (160) includes a cup (162) and a lid (166). Cup (162) defines a reservoir (164), which can be used to contain fluid within cup (162). Cup (162) defines a generally cylindrical shape that is sized to receive one or more tissue sample trays (100). Lid (166) generally corresponds to the cylindrical shape of cup (162). Lid (166) is further configured to be selectively fastened onto a top portion of cup (162). In the present example, lid (166) includes seals or other features configured to seal cup (162) relative to the exterior of cup (162). As described above, reservoir (164) is generally configured to hold fluid. Thus, lid (166) is corresponding configured to hold the fluid within cup (162).

As described above, jar (160) is generally filled with fluid. Thus, when tissue sample tray (100) is disposed within jar (160), tissue sample tray (100) is generally at least partially submerged in fluid. In the present example, fluid is generally configured to act as a preservative of tissue samples contained within tissue sample tray (100). By way of example only, one suitable preservative may include formalin. However, it should be understood that in other examples numerous alternative fluids as will be apparent to those of ordinary skill in the art in view of the teachings herein.

II. Exemplary Tissue Processing Cassette

Once tissue samples have been collected using biopsy device (10) or other similar devices described herein, it may be desirable to subject such tissue samples to further pathological analysis. To facilitate such analysis, such tissue samples may be subjected to a variety of processing steps described in greater detail below. During these processing steps, it may be desirable to dispose the collected tissue samples within a container or other device to help segregate and track the collected tissue samples relative to other tissue sample collected from the same or other patients as well as the same or other biopsy procedures.

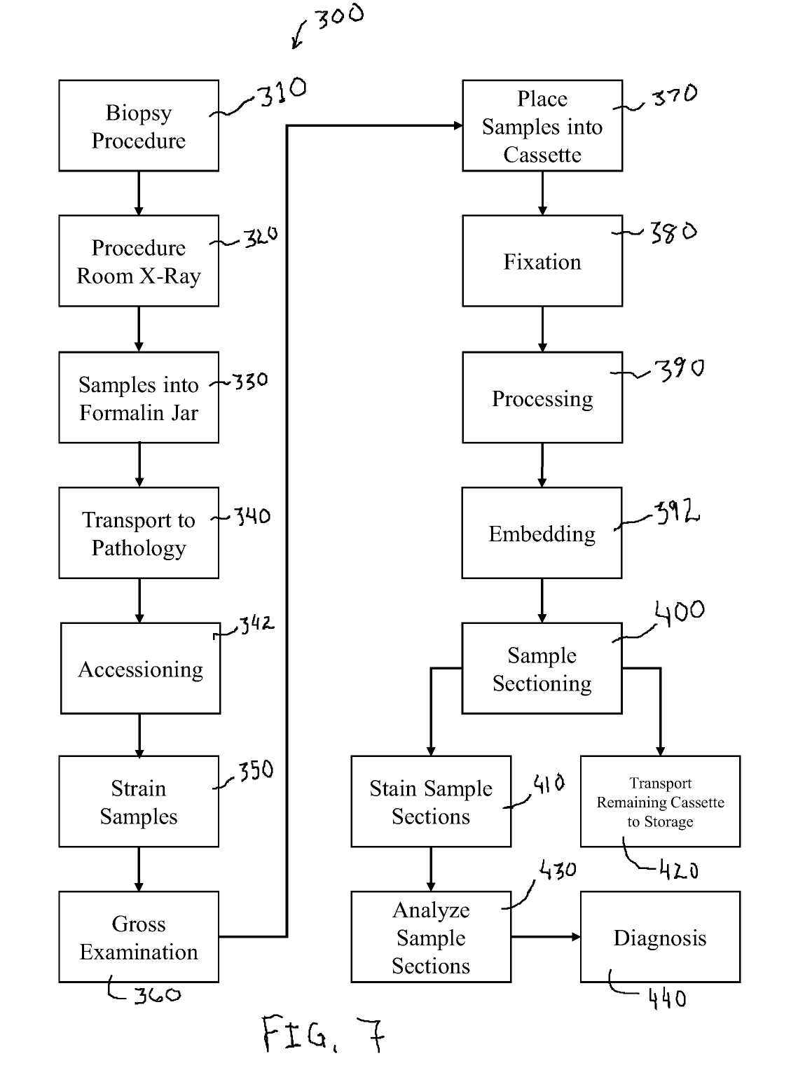

FIG. 6 shows an exemplary tissue processing cassette (200) that may be used in connection with biopsy device (10) to store and track tissue samples after collection via biopsy device (10). Tissue processing cassette (200) is generally configured to receive and enclose a plurality of tissue samples therein. As can be seen, tissue processing cassette (200) comprises a base (210) and a lid (230). Base (210) comprises a distal wall (212), a proximal wall (216), a pair of sidewalls (220), and a floor (222). Base (210) further includes a labeling surface (226) extending distally from distal wall (212).

Walls (212, 216, 220) are generally connected to form a rectangular pattern around floor (422). Each wall is generally solid, thereby forming a sample chamber (228) therein. As will be described in greater detail below, sample chamber (228) is generally configured to contain tissue samples within tissue processing cassette (200) when lid (230) is closed relative to base (210).

Distal wall (212) and proximal wall (216) each include a lid receiver (214, 218). Each lid receiver (214, 218) is generally configured to receive at least a portion of lid (230) to thereby selectively secure lid (230) to base (210). Although not shown, it should be understood that each lid receiver (214, 218) can include certain fastening features to facilitate securing lid (230) to base (210). As will be described in greater detail below, these fastening features generally facilitate a snap fit coupling mechanism. However, it should be understood that in other examples alternative coupling mechanisms may be used such as compression fit mechanisms, or any other suitable coupling mechanism as will be apparent to those of ordinary skill in the art in view of the teachings herein.

As described above, labeling surface (226) protrudes distally from distal wall (212). Labeling surface (226) is generally configured to receive a label to provide information to an operator related to the samples contained within tissue processing cassette (200). Although labeling surface (226) of the present example can receive a label (e.g., a pre-printed self-adhering label), it should be understood labeling surface (226) is also configured to permit direct printing of a label onto labeling surface (226). For instance, in some examples labels are laser etched onto labeling surface (226) using a printer configured to receive tissue processing cassette (200) and thereby print directly onto labeling surface (226). To facilitate such printing, it should be understood that labeling surface (226) can also be equipped with a colored coating that can be etched away by the printer described above.

Floor (222) includes a plurality of vents (224) arranged in an array across the surface of floor (222). Vents (224) are generally configured to promote the flow of fluid through floor (222), yet maintain tissue samples within sample chamber (228). To facilitate this configuration, vents (224) have a narrow rectangular form. In other examples, vents (224) can be configured with a variety of alternative shapes such as round, oval-shaped, square, and/or etc. Although vents (224) in the present example are arranged to uniformly occupy the entire surface of floor (222), it should be understood that in other examples vents (224) can be arranged in a variety of other ways. For instance, vents (224) can be isolated to a specific region or multiple regions of floor (222). Of course, other alternative arrangements for vents (224) will be apparent to those of ordinary skill in the art in view of the teachings herein.

Lid (230) comprises a cover portion (234) that is generally configured to engage base (210) to hold tissue samples within sample chamber (228) of base (210). Lid (230) further includes a lip (238) protruding from cover portion (234). Lip (238) extends around the perimeter of cover portion (234) defining a rectangular shape that corresponds to the rectangular shape defined by walls (212, 216, 220) of base (210). As will be understood, lip (238) is generally configured to fit within sample chamber (228) adjacent to each wall (212, 216, 220) to laterally secure and locate cover portion (234) relative to base (210) when lid (230) is in a closed position relative to base (210).

Like with floor (222) described above, cover portion (234) likewise includes a plurality of vents (236) arranged in an array across the surface of cover portion (234). Like vents (224) described above, vents (236) are generally configured to promote the flow of fluid through cover portion (234), yet maintain tissue samples within sample chamber (228). To facilitate this configuration, vents (236) have a narrow rectangular form. In other examples, vents (236) can be configured with a variety of alternative shapes such as round, oval-shaped, square, and/or etc. Although vents (236) in the present example are arranged to uniformly occupy the entire surface of cover portion (234), it should be understood that in other examples vents (236) can be arranged in a variety of other ways. For instance, vents (236) can be isolated to a specific region or multiple regions of cover portion (234). Of course, other alternative arrangements for vents (234) will be apparent to those of ordinary skill in the art in view of the teachings herein.

Lid (230) further comprises a proximal fastener (240) and distal fastener (242). Proximal fastener (240) is configured to engage lid receiver (218) of proximal wall (216), while distal fastener (242) is configured to engage lid receiver (214) of distal wall (212). Each fastener (240, 242) includes a tooth, lip, or other engagement feature that mates with a corresponding feature of each lid receiver (214, 218). As described above, this generally provides a snap fit coupling between each fastener (240, 242) and each lid receiver (216, 218) to selectively maintain lid (230) in the closed position.

Lid (230) is secured to proximal wall (216) of base (210) with an integral living hinge (232). Living hinge (232) permits pivoting of lid (230) relative to base (210) such that lid (230) may move between an open position (e.g., FIG. 6) and the closed position. This configuration permits tissue samples to be loaded into sample chamber (228) when lid (230) is in the open position. Lid (230) can then be pivoted to the closed position to secure the loaded tissue samples within sample chamber (228). To assist pivoting of lid (230), lid (230) further includes a manipulator (244) or thumb snap. Manipulator (244) generally protrudes distally from cover portion (234) to provide a gripping feature when lid (230) is in the closed position. This facilitates moving lid (230) from the closed position to the open position by providing a surface for an operator to grasp.

As described above, tissue samples may be subjected to various processing and or analysis steps after the tissue samples are collected with biopsy device (10) or other suitable devices. During such steps, tissue processing cassette (200) can be used to facilitate transport, tracking, and storage of the collected tissue samples. In particular, FIG. 7 shows a generally workflow associated with biopsy device (10) and tissue processing cassette (200) described above. It should be understood that the workflow (300) shown in FIG. 7 and the description herein is only exemplary and that various alternative procedural steps may be used in addition and/or in the alternative to the steps shown in FIG. 7. For instance, in some examples biopsy device (10) and/or tissue processing cassette (200) may be used in accordance with one or more of the teachings of U.S. Ser. No. 15/638,843, entitled “Integrated Workflow for Processing Tissue Samples from Breast Biopsy Procedures,” filed on Jun. 30, 2017, the disclosure of which is incorporated by reference herein.

In the workflow (300) shown in FIG. 7, tissue samples are collected during a biopsy procedure represented by box (310). During the biopsy procedure in box (310), biopsy device (10) may be used to collect a plurality of tissue samples into one or more tissue sample trays (100). Although the description above is primarily related to collection of tissue samples using a stereotactic biopsy procedure, it should be understood that various alternative procedures can be used such as ultrasonically guided procedures, Mill guided procedures, and/or etc. In addition, although the description above is primarily related to tissue sample collection using a multi-chamber-style tissue sample holder similar to tissue sample holder assembly (40), it should be understood that various alternative tissue sample collection devices may be used such as basket-style tissue sample holders. Alternatively, tissue samples can be collected without a tissue sample holder and may be merely plucked from a sample surface on a device similar to biopsy device (10).

Regardless of the particular process for collecting tissue samples, once tissue samples are collected, they may be subjected to procedure room x-ray as shown in box (320). During procedure room x-ray, an operator uses x-ray imaging in the procedure room to perform preliminary analysis on the collected tissue samples. During this stage, the collected tissue samples are primarily analyzed using x-ray imaging to determine if any one or more of the collected tissue samples include calcifications or other suspicious features identifiable via x-ray. After this preliminary analysis, more tissue samples can be acquired, if an operator is not satisfied with the preliminary analysis. Alternatively, an operator may be satisfied with the originally collected tissue samples and move to the next step in the procedure.

After an operator is satisfied with preliminary procedure room x-ray analysis, the operator may insert tissue sample tray (100) or just the tissue samples into jar (160). As described above, jar (160) may be filled with formalin or other fluids to preserve the collected tissue samples for storage and/or transport as represented by box (330). Jar (160) is then transported to a pathology laboratory so that the tissue samples can be subjected to further analysis as represented by box (340).

Once jar (160) is received by the pathology laboratory, the samples can be subjected to accessioning as represented by box (342). Accessioning (342) used herein refers to the process of documenting the chain of custody of the collected tissue samples. It should be understood that this may include a variety of steps. For instance, in some examples, jar (160) can include a label that can be used to store, present, display, or otherwise provide patient information. This label can be printed during or after the biopsy procedure described above and represented by box (310). The label can then be adhered to jar (160) prior to transport to pathology as represented by box (340). Once jar (160) is received by pathology, an operator can record, scan, or otherwise collect information form the label to track the chain of custody of the collected tissue samples contained within jar (160).

Once accessioning is complete, the collected tissue samples are strained from the fluid contained within jar (160) as represented by box (350). The collected tissue samples then undergo gross examination by an operator as represented by box (360). Gross examination can include visual inspection of the collected tissue samples, palpitation of the collected tissue samples, and/or manipulating the collected tissue samples into a desired position. Preliminary observations can then be documented in a written record by an operator. Such written records can then be accessioned with the label described above with respect to accessioning and box (342).

After gross examination or during gross examination, the collected tissue samples are inserted into one or more tissue sample processing cassettes similar to tissue processing cassette (200) described above as represented by box (370). For instance, in the context of tissue processing cassette (200), each collected tissue sample is generally laid on floor (222) of base (210) longitudinally between distal wall (212) and proximal wall (216). Lid (230) is then pivoted to the closed position to enclose the collected tissue samples within sample chamber (228) of base (210). To promote tracking of the collected tissue samples, the tissue processing cassette can be labeled at this stage by either direct printing or adhering a self-adhering label to a structure similar to labeling surface (226) described above. This label can include certain patient information corresponding to the label described above with respect to accessioning and box (342).

Once the collected tissue samples are disposed within a tissue processing cassette similar to tissue processing cassette (200), the collected tissue samples are subjected to fixation as represented by box (380). The term fixation used herein refers to the process of using a fixative to preserve specimen integrity and to maintain the shape of cells. Generally, this process involves submerging the collected tissue samples within a fixative. One common fixative is 10% neutral buffered formalin, although other fixatives can be used. The collected tissue samples can be maintained within the fixative for a predetermined period of time. Suitable periods of time can vary according to a variety of factors. However, under many circumstances, a suitable period of time can be approximately 6 hours. This period is generally sufficient to provide stabilization of the proteins in the collected tissue samples to substantially to prevent degeneration of the collected tissue samples.

After fixation is complete, the collected tissue samples are subjected to various chemical solutions during the processing step represented by box (390). During this process, multiple tissue processing cassettes may be loaded into a basket for bulk processing. Various chemicals are then applied, which may enter each tissue processing cassette via vents similar to vents (224, 236) described above. Various chemicals may be used during this process such as alcohols of various concertation levels. For instance, when alcohol is used, moisture is removed from each collected tissue sample rendering each collected tissue sample hard in texture and generally dehydrated.

Once processing is complete, the collected tissue samples are subjected to embedding process represented by box (392). During the embedding process, the collected tissue samples are surrounded by a histological wax. In one merely exemplary embedding process, the tissue samples are removed from the tissue processing cassette and placed into a metal tray or container. Prior to placement of the tissue samples within the metal tray, the metal tray can be partially filled with an initial amount of molten wax. Once the samples are placed in the metal tray, the metal tray is then filled with molten wax. The tissue processing cassette is then placed on the top of the metal tray with the underside of the cassette facing the tissue samples. Additional molten wax is then added through the cassette to bond with wax in the metal tray. During this process, the metal tray can be placed on a cold plate or other cold surface to provide relatively quick solidification of the wax. Once solidification is complete, the collected tissue samples and cassette can be removed from the metal tray. It should be understood that once the tissue samples are prepared in this manner, the tissue samples are generally preserved for indefinite storage at room temperature.

After the embedding process is complete, thin slices of each collected tissue sample are acquired as represented by box (400). Sample sectioning may be performed using a microtome machine. Such a machine uses precision blades to slice thin samples longitudinally from each collected tissue sample. The thin sections are then placed on slides for viewing under suitable visualization means such as optical microscopes.

Once the tissue sample sections are placed on a slide, the sections are subjected to staining as represented by box (410). The portion of the collected tissue samples that remain in the tissue processing cassette are transported to storage as represented by box (420). During the staining process, various chemical compounds are applied to the tissue sample sections. Each chemical compound may be configured to react to different tissue cells. For instance, some compounds may be configured to specifically react with cancer cells, thereby staining cancer cells with a distinctive color relative to other cells. Although not represented in FIG. 7, it should be understood that in some examples the staining process can include multiple stages of staining. For instance, in some examples staining can include primary staining followed by advanced staining.

Once staining is complete, the stained sample sections are analyzed by an operator using a microscope or other visualization means as represented by box (430). Based on this analysis a diagnosis may be generated as represented by box (440).

III. Exemplary Integrated Tissue Collection and Processing System

In some instances it may be desirable to combine certain elements of the tissue sample holder assembly (40) described above with the tissue analysis cassette (200) described above. For instance, manipulation of tissue samples generally risks degrading the quality of the tissue samples each time the tissue samples are manipulated due to the fragility of the tissue. Transferring tissue samples between elements like tissue sample tray (100) described above and tissue processing cassette (200) described above often result in at least some manipulation of the tissue samples being transferred. Thus, transferring tissue samples between various elements may be undesirable in certain circumstances because this can lead to degradation of tissue sample quality. It is therefore desirable to reduce the number of containers used to deposit tissue samples during the workflow (300) described above.

In addition to manipulation of tissue samples being generally undesirable, transferring tissue samples between different containers (e.g., tissue sample tray (100), tissue processing cassette (200)) can lead to mislabeling or tacking errors associated with tissue samples as the tissue samples progress through the workflow (300) described above. For instance, when tissue sample are transferred from tissue sample tray (100) to tissue processing cassette (200), incorrect patient information might be printed on tissue processing cassette (200). Another possibility is that an incorrect label may be placed on tissue processing cassette (200). Thus, transferring tissue samples between different containers also includes the risk of generating errors in tissue sample tracking. Accordingly, it is desirable to reduce the number of containers used in the workflow (300) described above to generally improve tissue sample integrity and reduce operator error.

Although various devices and methods are described below for reducing the number of containers used in the workflow (300) described above are described herein, it should be understood that various alternative configurations will be apparent to those of ordinary skill in the art in view of the teachings herein. For instance, some suitable alternative configurations may combine various features of one embodiment described below with various features of another alternative embodiment. Still other suitable alternative configurations may omit various features of one or more embodiments. Of course, other suitable configurations will be apparent to those of ordinary skill in the art in view of the teachings herein.

A. Exemplary Cassette Assembly

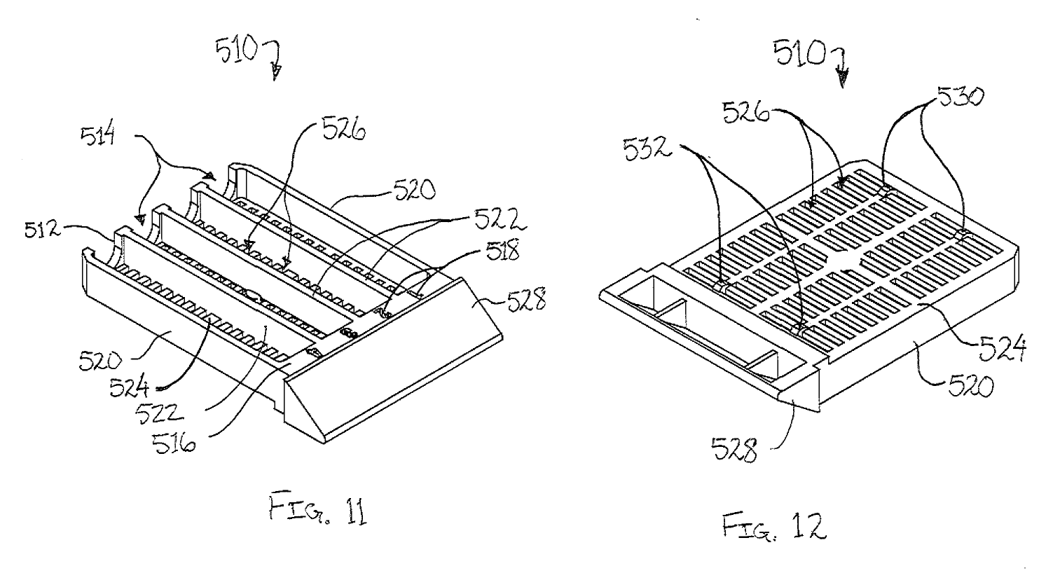

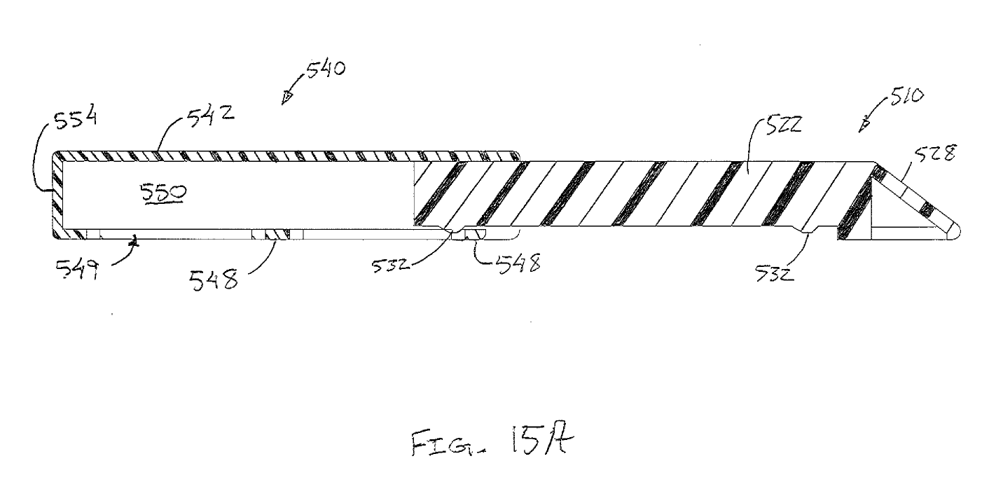

FIGS. 8-14 show and exemplary cassette assembly (500) that may be used with biopsy device (10) described above. As will be understood, cassette assembly (500) is generally configured to receive tissue samples during a biopsy procedure and then continue to contain the tissue samples after the biopsy procedure and through various sample analysis procedures. In other words, cassette assembly (500) can be used in lieu at least tissue processing cassette (200) described above. In addition, cassette assembly (500) may also be used in lieu of tissue sample tray (100), as will be described in greater detail below. However, in some uses, cassette assembly (500) may merely be supplementary to tissue sample tray (100) or other analogous features (e.g., a bulk sample basket).

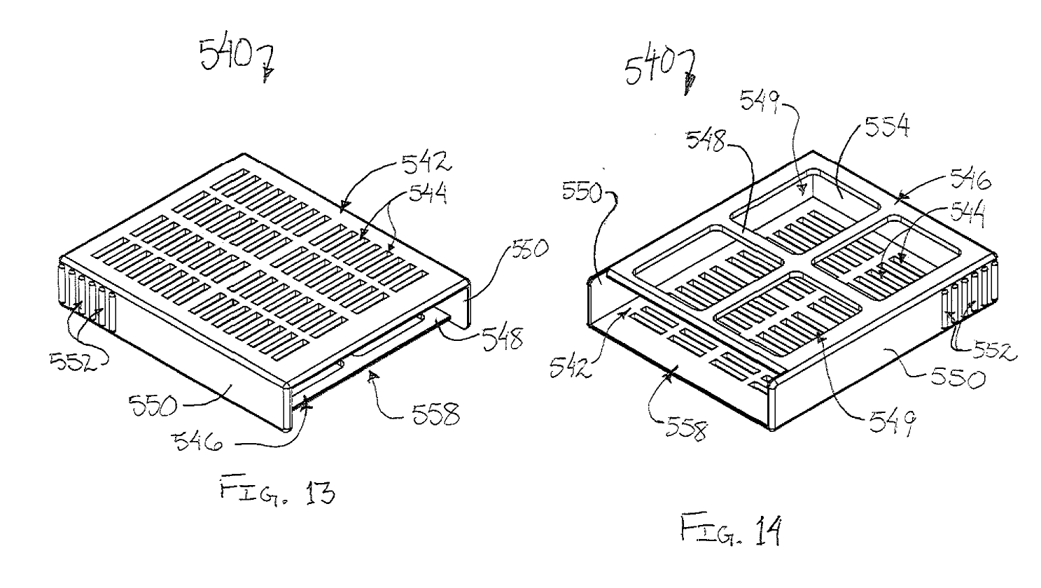

As best seen in FIG. 10, cassette assembly (500) includes a cassette tray (510) and a cover (540). Cassette tray (510) comprises a distal wall (512), a proximal wall (516), a pair of sidewalls (520) extending between distal wall (512) and proximal wall (516), and a floor (524) positioned below walls (512, 516, 520). Distal wall (512) includes a plurality of openings (514) evenly spaced laterally across the face of distal wall (512). As will be described in greater detail below, each opening (514) is generally configured to receive a tissue sample. Proximal wall (516), by contrast, is solid. However, unlike distal wall (512), proximal wall (516) includes a plurality of indicia (518) on the upper surface of proximal wall (516). In the present example, indicia (518) form a plurality of unique numerical identifiers. In other examples, indicia (518) may take a variety of forms such as letters or discrete shapes or symbols.

Walls (512, 516, 520) are interconnected to form the outer perimeter of cassette tray (510). Internally, cassette tray (510) includes a plurality of inner divider walls (522) extending longitudinally from distal wall (512) to proximal wall (516). Each inner divider wall (522) is positioned parallel relative to sidewalls (520) an equal distance apart to define a plurality of discrete sample chambers (523). Each sample chamber (523) is generally configured to hold a single tissue sample severed by biopsy device (10). Although the present example includes four discrete sample chambers (523), it should be understood that in other examples any other suitable number of sample chambers (523) can be used. In such examples, it should be understood that each sample chamber (523) can be configured for receiving more than a single tissue sample as with sample chambers (523) in the present example.

Floor (524) is positioned below walls (512, 516, 520, 522). In the present example, each wall (512, 516, 520, 522) is integral with each wall. However, in other examples one or more of each wall (512, 516, 520, 522) can be separate from floor (524) and attached with adhesive or some form of mechanical fastening. Floor (524) includes a plurality of vents (526). Vents (526) are generally configured to promote the flow of fluid through floor (524), yet maintain tissue samples within each sample chamber (523). To facilitate this configuration, vents (526) have a narrow rectangular form. In other examples, vents (526) can be configured with a variety of alternative shapes such as round, oval-shaped, square, and/or etc. Although vents (526) in the present example are arranged to uniformly occupy the entire surface of floor (524), it should be understood that in other examples vents (526) can be arranged in a variety of other ways. For instance, vents (526) can be isolated to a specific region or multiple regions of floor (524). Of course, other alternative arrangements for vents (526) will be apparent to those of ordinary skill in the art in view of the teachings herein.

Floor (524) is opposite to an open space above each sample chamber (523). Thus, the upper portion of cassette tray (510) is generally open. Because of this, tissue samples may be deposited into each sample chamber (523) through openings (514) in distal wall (512) or through the open upper portion of cassette tray (510). As will be described in greater detail below, tissue samples are generally contained within each sample chamber (523) once cassette tray (510) is received within cover (540).

Cassette tray (510) further includes a labeling portion (528) protruding proximally from proximal wall (516). Labeling portion (528) generally defines a triangular or wedge shape that provides a flat smooth surface for printing or otherwise adhering a label to the surface of labeling portion (528). As similarly described above with respect to labeling surface (226) of tissue processing cassette (200), labeling portion (528) is generally configured to provide readily accessible patient information to an operator to aid with tracking of tissue samples as they progress through the biopsy and sample analysis procedure.

Unlike labeling surface (226) described above with respect to tissue processing cassette (200), at least a portion of labeling portion (528) is generally oversized relative to the height of sidewalls (520) or the lateral length of proximal wall (516). This feature generally provides a blocking or sealing feature for cassette tray (510) to promote the flow of fluid through cassette tray (510). As will be described in greater detail below, cassette tray (510) is generally insertable into cover (540) or other components. When inserted into cover (540) or other suitable components, labeling portion (528) blocks at least a portion of cover (540) and/or other components to force fluid flow through vents (526) rather than other features of cassette tray (510).

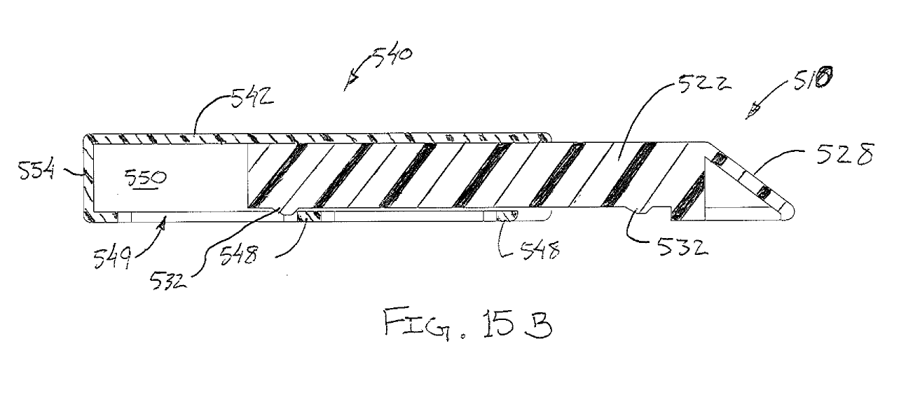

As best seen in FIG. 12, cassette tray (510) further comprises a plurality of detents (530, 532) disposed on the underside of floor (524). As can be seen, cassette tray (510) comprises a pair of distal detents (530) and a pair of proximal detents (532). Distal detents (530) are positioned approximately adjacent to distal wall (512), while proximal detents (532) are positioned approximately adjacent to proximal wall (516). As will be described in greater detail below, each pair of detents (530, 532) is positioned to provide temporary or selective locking of cassette tray (510) at various positions relative to cover (540) when cassette tray (510) is inserted into cover (540). Although detents (530, 532) are shown as having a generally rectangular shape with rounded corners, it should be understood that various alternative shapes may be used in other examples. For instance, detents (530, 532) can be hemispherical, oval-shaped, triangular, and/or etc.

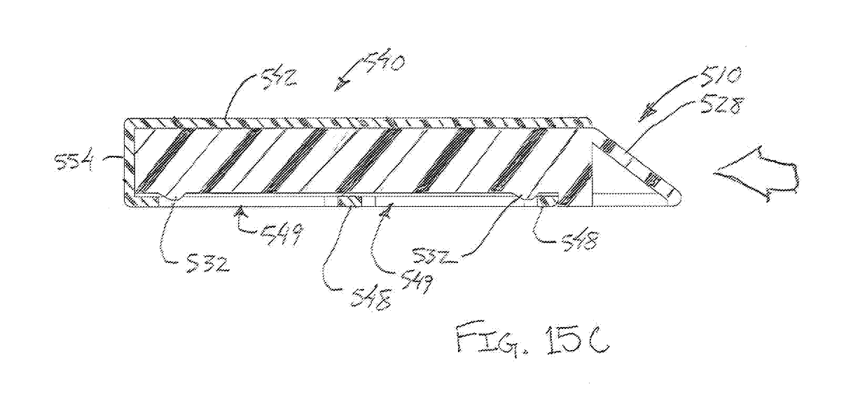

FIGS. 13 and 14 show cover (540) in greater detail. As can be seen, cover (540) comprises a filter portion (542), a support portion (546), and a plurality of walls (550, 554) extending between the filter portion (542) and the support portion (546). Filter portion (542) is similar to floor (524) described above in that filter portion (542) includes a plurality of vents (544) arranged in an array about the surface of filter portion (542). Vents (544) are generally configured to promote the flow of fluid through filter portion (542), yet maintain tissue samples within each sample chamber (523) of cassette tray (510) when cassette tray (510) is inserted into cover (540). To facilitate this configuration, vents (544) have a narrow rectangular form. In other examples, vents (544) can be configured with a variety of alternative shapes such as round, oval-shaped, square, and/or etc. Although vents (544) in the present example are arranged to uniformly occupy the entire surface of filter portion (542), it should be understood that in other examples vents (544) can be arranged in a variety of other ways. For instance, vents (544) can be isolated to a specific region or multiple regions of filter portion (542). Of course, other alternative arrangements for vents (544) will be apparent to those of ordinary skill in the art in view of the teachings herein.

Unlike filter portion (542), support portion (546) omits structures similar to vents (544). Instead, support portion (546) includes a support structure (548) defining a plurality of open spaces (549). As will be understood, support portion (546) is generally adjacent to floor (524) of cassette tray (510) when cassette tray (510) is inserted into cover (540). Thus, including structures similar to vents (544) is not entirely necessary due to the presence of vents (526) in floor (524) of cassette tray (510). However, it should be understood that in some examples support portion (546) may include structures similar to vents (544).

Support structure (548) forms a generally cross-shaped pattern in support portion (546). This structure is generally configured to provide rigidity to cover (540) and is further configured to hold cassette tray (510) within cover (540) when cassette tray (510) is disposed within cover (540). Although support structure (548) forms a generally cross-shaped pattern in the present example, it should be understood that in other examples support structure (548) can take on a variety of other forms. For instance, in some examples support structure (548) can have a lath-shaped structure. In other examples, support structure (548) can have a lattice shaped structure. In still other examples, support structure (548) can be formed of a plurality of concentric circles, or any other configuration as will be apparent to those of ordinary skill in the art in view of the teachings herein.

Support structure (548) is further configured to interact with detents (530, 532) of cassette tray (510). As will be described in greater detail below, cassette tray (510) is generally insertable into cover (540) at a plurality of discrete positions relative to cover (540). During insertion, detents (530, 532) of cassette tray (510) interact with support structure (548) to bias cassette tray (510) towards each discrete position. Due to the cross-shaped pattern of support structure (548), support structure (548) provides three discrete positions of cassette tray (510) relative to cover (540). Of course, in other examples where support structure (548) defines a different shape, support structure (548) can provide more or less discrete positions for cassette tray (510) relative to cover (540).

With the cross-shaped pattern of support portion (546), support portion (546) defines four total open spaces (549). Open spaces (549) are generally configured to promote fluid flow through cover (540) between vents (544) of filter portion (542) and open spaces (549). As will be described in greater detail below, this permits fluid to flow through cassette tray (510) when cassette tray (510) is disposed within cover (540).

As described above, cover (540) includes a plurality of walls (550, 554) extending between filter portion (542) and support portion (546). Walls (550, 554) include a pair of sidewalls (550) and a distal wall (554). Sidewalls (550) and distal wall (554) are both solid to generally promote rigidity of cover (540). Each sidewall (550) includes a plurality of grips (552), which promote manipulation of cover (540) by an operator. Walls (550, 554) together with filter portion (542) and support portion (546) together are configured to define an enclosure for cassette tray (510) that holds tissue samples within cassette tray (510), while permitting fluid to flow through cassette tray (510).

Opposite distal wall (554), filter portion (542), support portion (546), and sidewalls (550) define a proximal opening (558). Proximal opening (558) is generally configured to receive at least a portion of cassette tray (510) such that cassette tray (510) may be inserted into cover (540). Although proximal opening (558) is shown in the present example as having a generally rectangular shape, it should be understood that proximal opening (558) is generally a function of the shape of cover (540) and cassette tray (510). Thus, in examples where cassette tray (510) and/or cover (540) take on different shapes, proximal opening (558) may also be correspondingly different.

FIGS. 15A-15C show an exemplary insertion of cassette tray (510) into cover (540). As will be described in greater detail below, insertion of cassette tray (510) into cover (540) generally occurs during a biopsy procedure after tissue samples have been collected by biopsy device (10) and inserted into cassette tray (510). As can be seen in FIG. 15A, the distal end of cassette tray (510) is initially inserted into proximal opening (558) of cover (540). As cassette tray (510) is inserted into proximal opening (558) of cover (540), distal detents (530) engage support structure (548) of support portion (546). Further insertion of cassette tray (510) into cover (540) causes distal detents (530) to flex over support structure (548) before releasing into the open spaces (549) that are oriented proximally on support portion (546).

Once distal detents (530) are disposed within the open spaces (549) that are oriented proximally on support portion (546) as shown in FIG. 15A, cassette tray (510) is generally removably secured within cover (540). In this context, “removably secured” refers to how cassette tray (510) is generally restricted from being pulled proximally out of cover (540). However, it should be understood that cassette tray (510) may still be pulled proximally out of cover (540) if a sufficient amount of force is applied to flex distal detents (530) upwardly onto support structure (548). At the same time, it should be understood that cassette tray (510) remains freely translatable in the distal direction such that cassette tray (510) can be advanced further into cover (540). In the position shown in FIG. 15A, cassette tray (510) can be optionally used by an operator while partially disposed within cover (540). By way of example only, this may be desirable for positioning or repositioning tissue samples within cassette tray (510).

Once cassette tray (510) is initially inserted into cover (540), an operator can insert cassette tray (510) further into cover (540) in the proximal direction towards the position shown in FIG. 15B. Cassette tray (510) is freely insertable in the proximal direction until distal detents (530) again engage support structure (548). Once distal detents (530) are engaged with support structure (548), an operator can apply a force to cassette tray (510) or cover (540) to flex distal detents (530) onto support structure (548) (or to flex support structure (548) out of the way of distal detents (530)).

Once distal detents (530) are clear of support structure (548), distal detents (530) will flex back to their original position and into the open spaces (549) oriented distally on cover (540). Once distal detents (530) are disposed within the open spaces (549) that are oriented distally on support portion (546) as shown in FIG. 15B, cassette tray (510) is generally removably secured within cover (540). Similar to the context above, “removably secured” here refers to how cassette tray (510) is generally restricted from being pulled proximally out of cover (540). However, it should be understood that cassette tray (510) may still be pulled proximally out of cover (540) if a sufficient amount of force is applied to flex distal detents (530) upwardly onto support structure (548). At the same time, it should be understood that cassette tray (510) remains freely translatable in the distal direction such that cassette tray (510) can be advanced further into cover (540). In the position shown in FIG. 15B, cassette tray (510) can be optionally used by an operator while partially disposed within cover (540). By way of example only, this may be desirable for positioning or repositioning tissue samples within cassette tray (510).

Once cassette tray (510) is inserted into cover (540) to the position shown in FIG. 15B, an operator may desire to insert cassette tray (510) fully into cover (540). To insert cassette tray (510) fully into cover (540), an operator may move cassette tray (510) distally relative to cover (540) towards the position shown in FIG. 15C. As cassette tray (510) is moved distally relative to cover (540), proximal detents (532) will engage support structure (548) of cover. At this point, an operator can apply a force to either cassette tray (510) or cover (540) that is sufficient to flex proximal detents (532) upwardly and onto support structure (548) (or flex support structure (548) out of the way of proximal detents (532)). Cassette tray (510) can then proceed further distally until proximal detents (532) flex downwardly to their original position and into the open spaces (549) oriented proximally on cover (540) as shown in FIG. 15C.

Once cassette tray (510) is positioned relative to cover (540) as shown in FIG. 15C, further distal movement of cassette tray (510) is prevented by engagement between distal wall (512) of cassette tray (510) and distal wall (554) of cover (540). In addition, as described above, labeling portion (528) is generally oversized relative to the dimensions of proximal wall (516) of cassette tray (510) and sidewalls (520) of cassette tray (510). Accordingly, labeling portion (528) can also act to stop further distal movement of cassette tray (510) by engagement between labeling portion (528) and support portion (546), sidewalls (550), and filter portion (542) of cover (540). In addition, it should be understood that in some contexts filter portion (542) can also act as a seal to seal proximal opening (558) of cover (540) relative to the exterior of cover (540). In such circumstances, this sealing can act to force fluid through vents (526, 544) rather than proximal opening (558).

B. Exemplary Tissue Sample Holder Assembly with Imaging System

In some examples it may be desirable to use cassette tray (510) in connection with biopsy device (10) such that tissue samples are collected directly into cassette tray (510) rather than into a structure similar to tissue sample tray (100) described above. Because cassette tray (510) includes a generally rigid structure, it should be understood that cassette tray (510) is generally not insertable directly into rotatable member (44) described above. Instead, it may be desirable to replace tissue sample holder assembly (40) with an alternative tissue sample holder assembly to facilitate use of cassette tray (510) directly with biopsy device (10). As described above, tissue sample holder assembly (40) is generally configured to be completely removable from probe assembly (20) of biopsy device (10). Thus, a suitable alternative tissue sample holder assembly may be used in lieu of tissue holder assembly (40), provided certain vacuum and tissue sample collection couplings remain consistent between the suitable alternative tissue sample holder assembly and tissue sample holder assembly (40).

It may be further beneficial to immediately examine a recently biopsied tissue specimen through certain imaging modalities to thereby quickly analyze and assess the tissue properties. However, an operator may be limited in how quickly the tissue sample can be analyzed by an imaging device due to the time elapsed extracting the tissue sample from the tissue sample holder assembly, positioning the tissue sample into an examination container, and subsequently inserting the examination container into an imaging system to produce images of the specimen for analysis. A tissue sample holder assembly that is adapted to directly associate with an imaging system may be beneficial to reduce the amount of time and effort required to analyze a tissue sample during a biopsy procedure. Furthermore, being able to take an immediate image of a tissue specimen that was recently biopsied from a patient allows an operator to confirm whether the targeted tissue was successfully acquired at each instance of tissue extraction, thereby reducing the number of tissue samples extracted from the patient.

In biopsy devices such as device (10) described above, it may be beneficial to configure the components of the device, such as an alternate tissue sample holder that facilities the use of cassette tray (510), to cooperate with certain imaging modalities to thereby simplify the process for an operator to obtain and review graphical representations or other images of the biopsied tissue specimen. This practice may eliminate several intermediate steps required in generating images of a biopsied specimen and thus maximize the effectiveness of analyzing the characteristics of a tissue sample of a patient. It may be further desirable to integrate the imaging system with a tissue sample holder assembly into a single assembly, while in other instances it may be desirable to adapt the tissue sample holder assembly to function in association with a separate imaging modality.

FIGS. 16-21 show an exemplary alternative tissue sample holder assembly (600) that may be used with biopsy device (10) in lieu of tissue sample holder assembly (40) described above. As best seen in FIG. 16, tissue sample holder assembly (600) comprises a coupler (610), a rotatable member (620), and a sensor (680). Coupler (610) comprises a generally circular-shaped body (612) with a sealing lip (614), a pair of bayonet connectors (616), and a plurality of grips (618). Bayonet connectors (616) are configured to receive a pair of bayonet pins (not shown) of probe assembly (20) to selectively couple coupler (610) to probe assembly (20). Thus, bayonet connectors (616) and the bayonet pins of probe assembly (20) form a standard bayonet coupling assembly to selectively secure coupler (610) to probe assembly (20). In this configuration, circular-shaped body (612) is generally rotatable relative to probe assembly (20) to lock and unlock coupler (610) relative to probe assembly (20). To assist an operator with rotation of circular-shaped body (612), coupler (610) includes grips (618) to enhance grip of circular-shaped body (612) during locking and unlocking. Although the present example uses a bayonet coupling to secure coupler (610) to probe assembly (20), it should be understood that in other examples various alternative coupling features can be used as will be apparent to those of ordinary skill in the art in view of the teachings herein.

Although not shown, in some versions tissue sample holder assembly (600) may be connected to biopsy device (10) through a remote tissue collection assembly that is configured to permit tissue sample holder assembly (600) to be remotely positioned at a location adjacent to an operator while biopsy device (10) remains positioned adjacent to the biopsy site where a patient is located. In this instance, the remote tissue collection assembly is configured to transport the tissue samples extracted by biopsy device (10) from the biopsy site to the remote location of tissue sample holder assembly (600) through a series of transport conduits. The remote positioning of tissue sample holder assembly (600) relative to biopsy device (10) may be beneficial to allow an operator to visually inspect the tissue samples as they are extracted by positioning tissue sample holder assembly (600) nearby rather than directly coupled to biopsy device (10) adjacent the biopsy site. Examples of assemblies and methods that may be used to remotely position tissue sample holder assembly (600) from biopsy device (10) are described in U.S. App. No. 62/607,698, entitled “Remote Tissue Collection Indexing and Processing Cassette,” filed on Dec. 19, 2017.

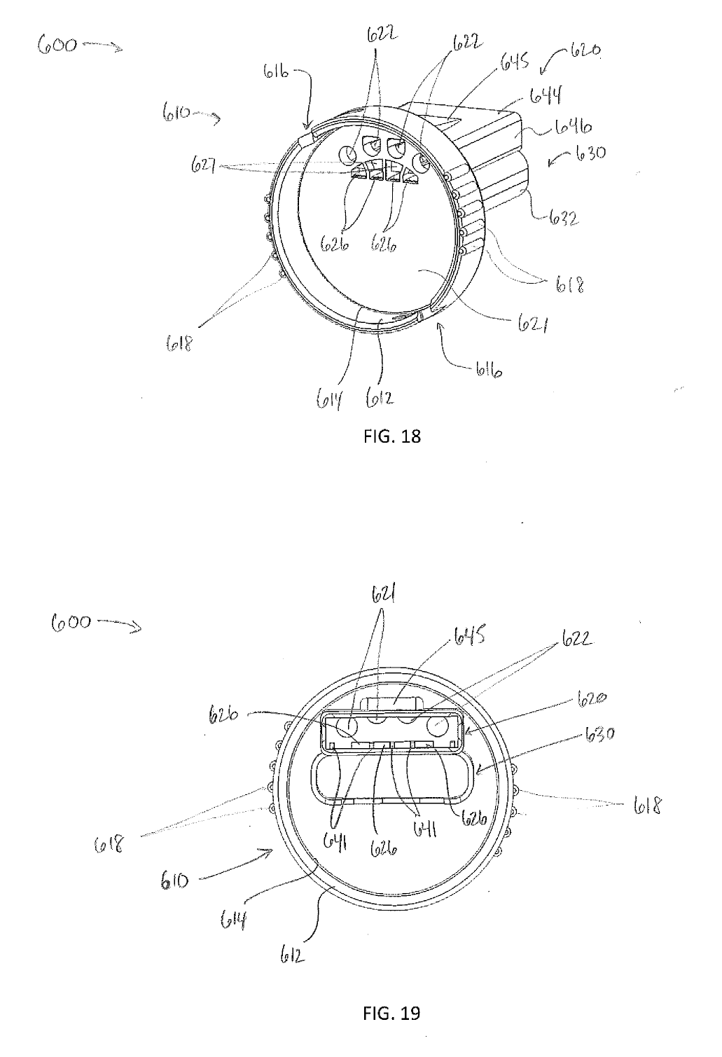

Rotatable member (620) is generally configured to receive cassette tray (510) and position cassette tray (510) relative to probe assembly (20) to thereby collect a tissue sample within each sample chamber (523) of cassette tray (510). Rotatable member (620) is also generally configured to receive sensor (680) and position sensor (680) relative to cassette tray (510) to thereby generate images of the collected tissue samples within each sample chamber (523) of cassette tray (510). Rotatable member (620) comprises a circular base (621), a sensor manifold (630) or mount protruding proximally from base (621), and a cassette manifold (640) or mount, also protruding from base (621). The circular shape of base (621) is generally configured for receipt within coupler (610) such that at least a portion of base (621) abuts sealing lip (614) of coupler (610). Accordingly, as best seen in FIGS. 17 and 18, sealing lip (614) is configured to engage circular base (621) of rotatable member (620) to seal rotatable member (620) relative to coupler (610) and probe assembly (20). In addition, sealing lip (614) is configured to permit rotation of base (621) of rotatable member (620) relative to coupler (610) and probe assembly (20). As will be described in greater detail below, this rotation permits cassette tray (510) to be moved relative to probe assembly (20) such that a single tissue sample can be collected within each sample chamber (523) of cassette tray (510).

As further seen in FIG. 17, manifold (640) comprises a lower wall (642), an upper wall (644), and a pair of sidewalls (646) extending between the lower wall (642) and the upper wall (646). Walls (642, 644, 646) together define a generally rectangular box that is configured to receive cassette tray (510). Walls (642, 644, 646) further define an inner chamber (652) that is large enough to accommodate cassette tray (510), while also providing fluid flow through manifold (640). Upper wall (644) includes a raised portion (645) that is generally hollow such that a portion of inner chamber (652) is defined by raised portion (645). As will be described in greater detail below, raised portion (645) is generally configured to receive tissue samples axially relative to the longitudinal axis of rotatable member (620) and direct tissue samples downwardly into cassette tray (510). Although raised portion (645) is shown as a single discrete part that is integral with upper wall (644), it should be understood that in other examples raised portion (645) can be a separate part, formed of more than one part, or a combination of both.

Sensor manifold (630) generally defines a hollow curved protrusion protruding proximally from base (621) defined by lower wall (642) and a pair of sidewalls (632). Sensor manifold (630) is generally sized and shaped to receive sensor (680) to thereby attach sensor (680) relative to cassette manifold (640). In particular, sensor manifold (630) is positioned below manifold (640) such that sensor (680) is received directly below cassette tray (510), as seen in FIG. 16. As will be described in greater detail below, sensor manifold (630) further includes a flexible detent (634) configured to releasably engage sensor (680) to thereby securely attach sensor (680) to sensor manifold (630).

As best seen in FIG. 18, rotatable member (620) communicates with probe assembly (20) via a plurality of openings (622, 626) defined by and extending axially through circular base (621). In particular, rotatable member (620) comprises a plurality of access openings (622) and a plurality of vacuum openings (626). The number of openings (622, 626) on rotatable member (620) corresponds to the number of chambers (523) in cassette tray (510) such that any tissue samples inserted into manifold (640) by a particular access opening (622) will be delivered and deposited within a respective chamber (523) of cassette tray (510). In the present example, circular base (621) defines four access openings (622) and four vacuum openings (626) in accordance with the four sample chambers (523) of cassette tray (510) as described above. In other words, each chamber (523) of cassette tray (510) communicates with the cutter of biopsy device (10) via a respective access opening (622). Although not shown, it should be understood that manifold (640) may comprise greater or fewer access openings (622) and vacuum openings (626) as will be apparent to those of ordinary skill in the art.

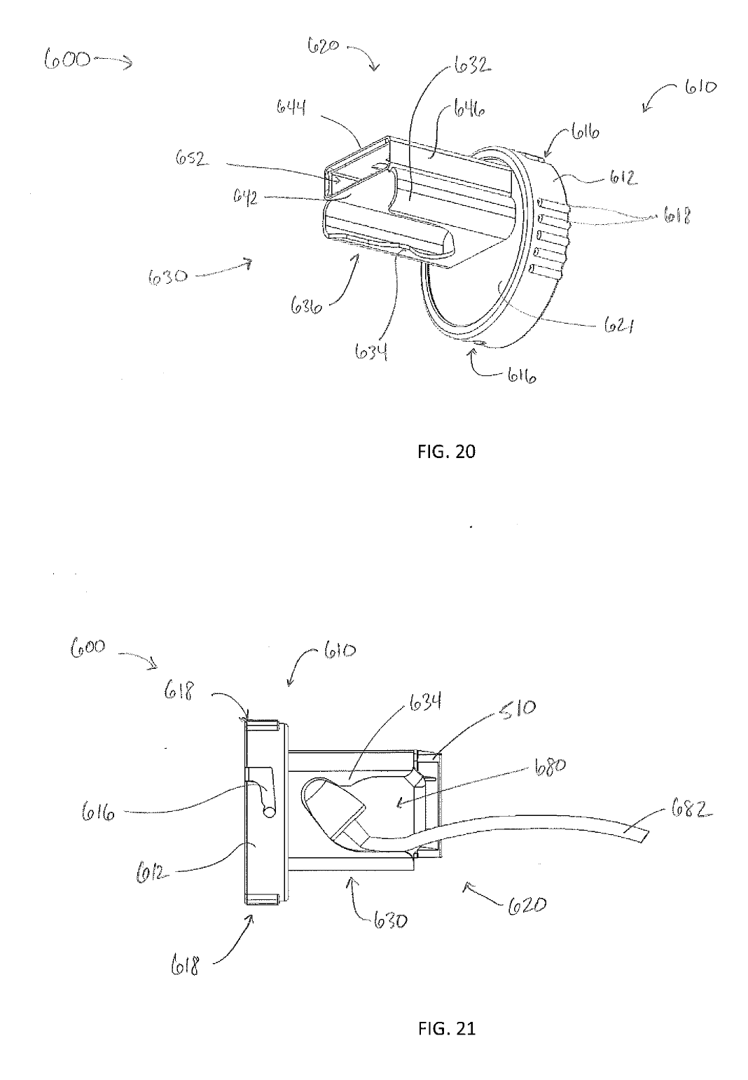

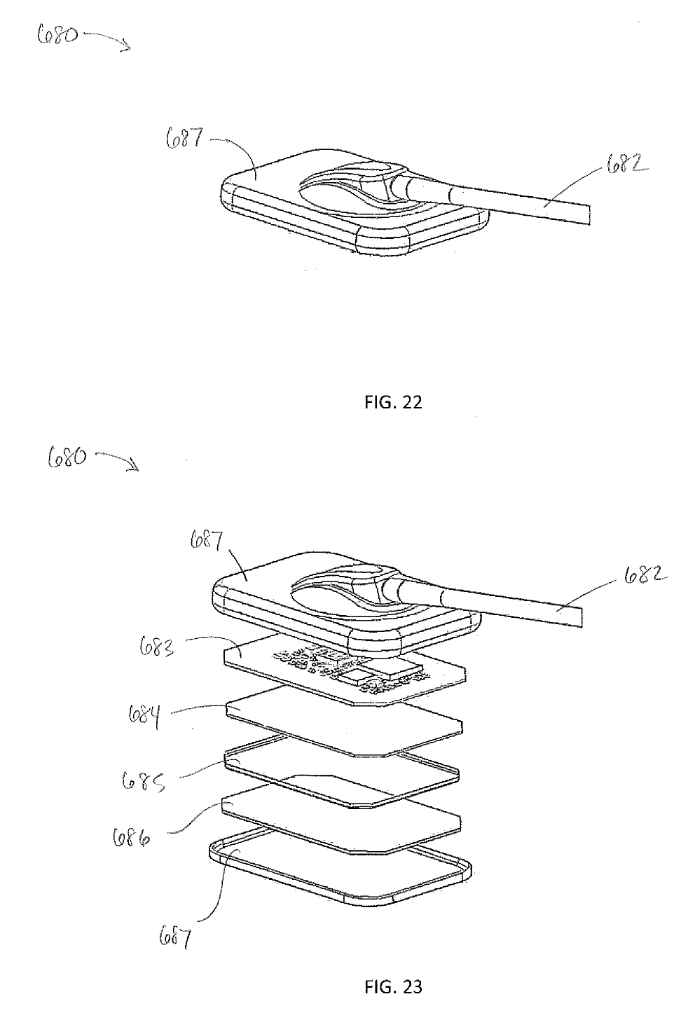

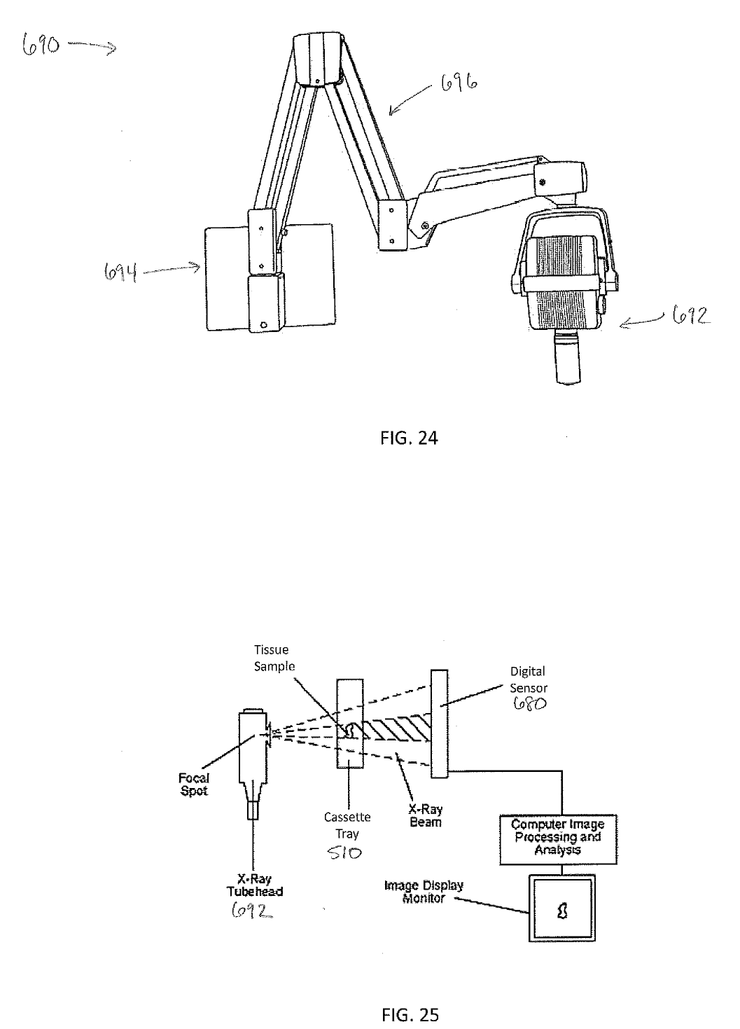

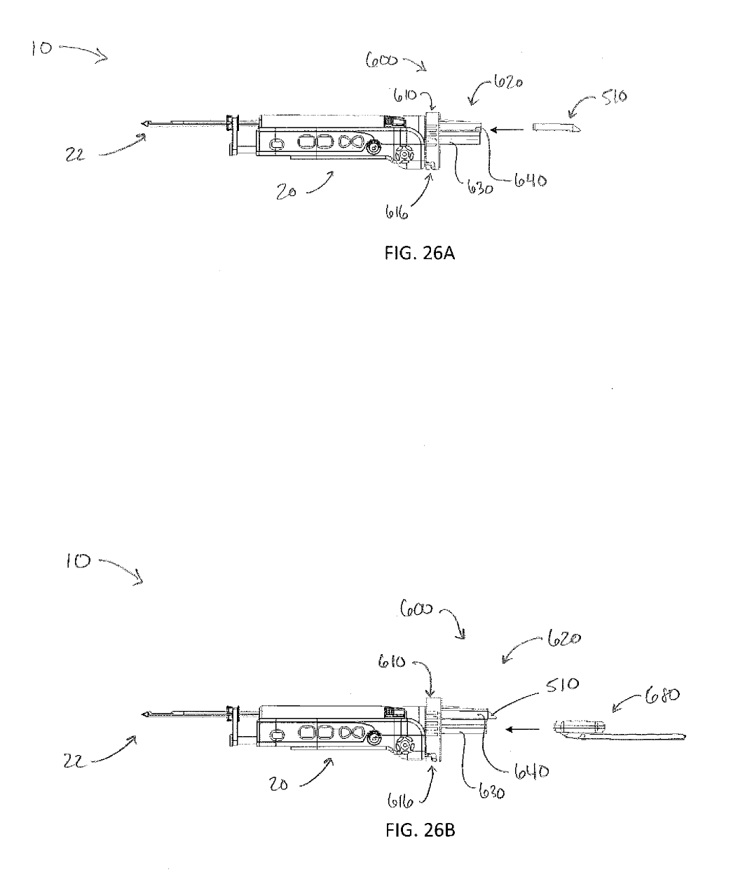

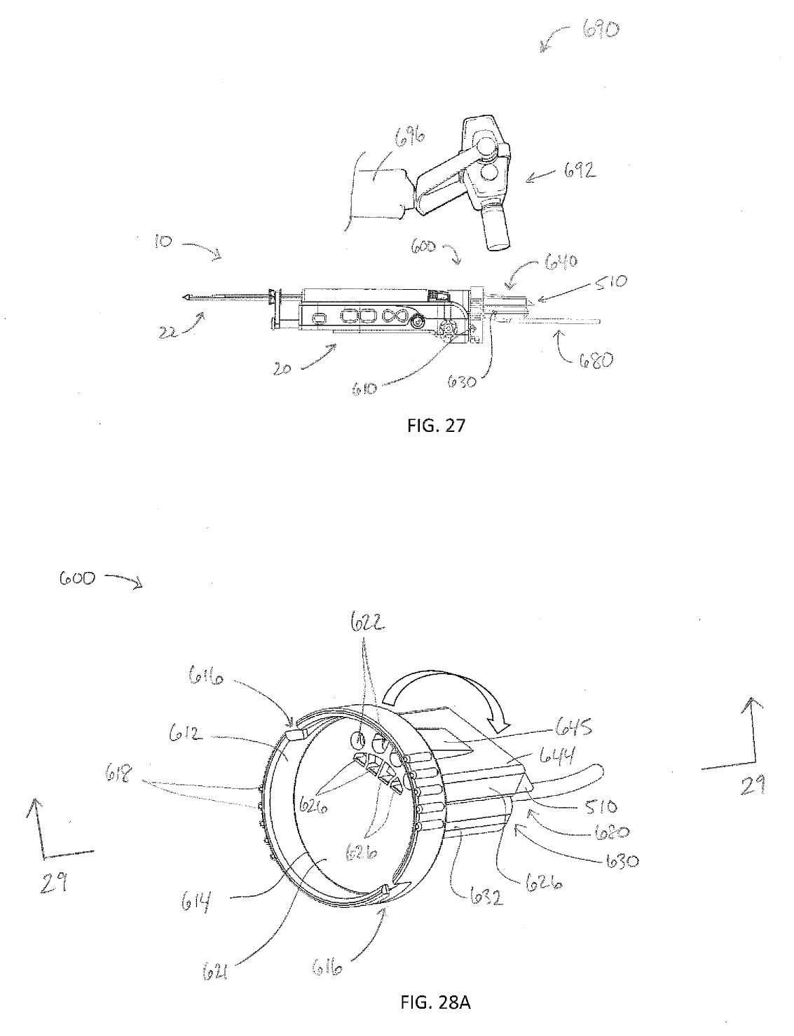

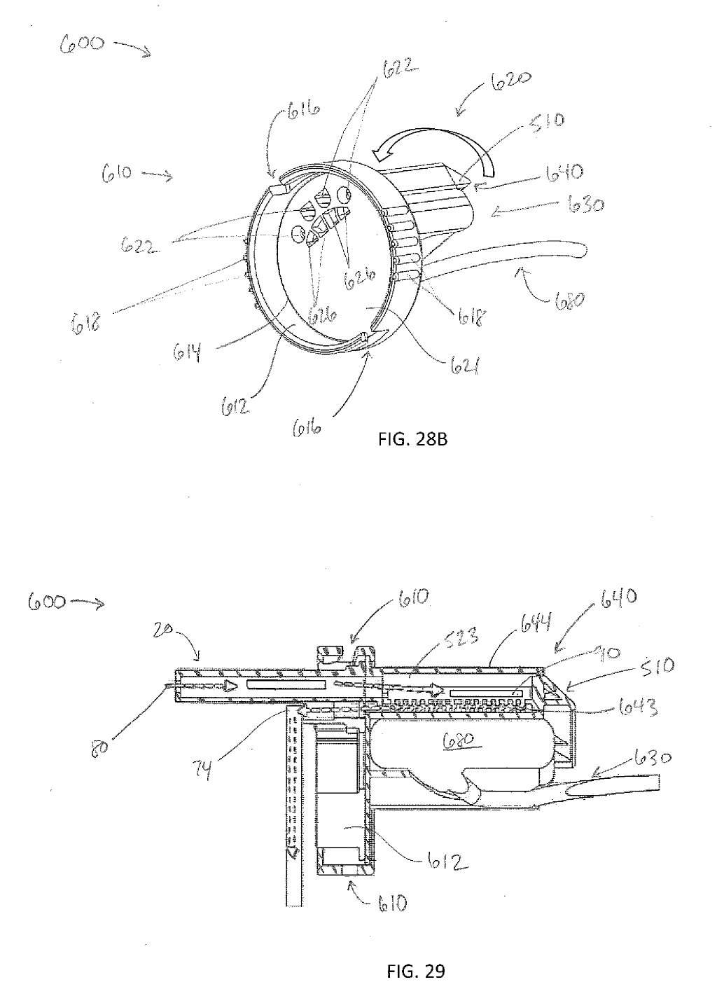

Of the four access openings (622), circular base (621) includes a corresponding vacuum opening (626). Access openings (622) are configured to provide fluid communication between probe assembly (20) and inner chamber (652) of manifold (640). Access openings (622) include respective channels that commence at opening (622) formed at circular base (621) and extend through manifold (640) at four distinct locations. In particular, the positions of access openings (622) correspond to distinct rotational orientations of rotatable member (620) relative to probe assembly (20). In other words, access openings (622) serve as tissue receiving channels that are orientated about manifold (620) at various predetermined locations that correspond to a respective portion within inner chamber (652) of manifold (640), as seen in FIG. 19. Access openings (622) are integrally formed within manifold (640) and are configured to align with channels (523) of cassette tray (510) when cassette tray (51) is received within manifold (640).

Access openings (622) are generally configured to individually receive tissue samples from the cutter of biopsy device (10) through probe assembly (20) such that only one access opening (622) maintains fluid communication with probe assembly (20) at a given moment depending on the particular rotational alignment of rotatable member (620) relative to probe assembly (20). As will be described in greater detail below, manifold (640) of rotatable member (620) can be rotated to align an access opening (622) with probe assembly (20) to thereby establish fluid communication between the cutter of biopsy device (10) and a particular channel (523) of cassette tray (510) that is aligned with that access opening (622). In other words, each respective access opening (622) aligns with a particular channel (523) of cassette tray (510) contained within inner chamber (652) such that access opening (622) can be used to provide a particular channel (523) access to the biopsy site when rotated to align with the cutter of biopsy device (10).

Rotatable member (620) further includes vacuum openings (626) extending into inner chamber (652) and positioned below each access openings (622), respectively, as seen in FIG. 19. Vacuum openings (626) include a respective channel (627) extending upwardly from vacuum opening (626) and toward a corresponding access opening (622). As best seen in FIG. 18, channels (627) are integral with rotatable member (620) such that channels (627) are cut into the distal end of rotatable member (620) at an adequate depth to provide communication between vacuum openings (626) and a vent port (not shown) of probe assembly (20). In particular, channels (627) are generally configured to correspond with the position of the vent port of probe assembly (20) when rotatable member (620) is rotated to align a particular access opening (622) with the cutter of biopsy device (10). In other words, channels (627) and vacuum openings (626) rotate simultaneously with rotatable member (620) such that when an access opening (622) arrives in alignment with the cutter of biopsy device (10) a corresponding channel (627) aligns with the vent port of probe assembly (20). In this instance, channel (627) establishes communication between the vent port of probe assembly (20) and vacuum opening (626) of rotatable member (620) to thereby allow for a vacuum to be formed within manifold (620).

As seen, channels (627) may comprise a straight, angular, or irregular shape and/or alignment in correspondence to the respective locations of access openings (622) and vacuum openings (626). The varying configurations of channels (627) allow vacuum openings (626) to be aligned in a straight line along the distal end of rotatable member (620), as best seen in FIG. 19. The straight alignment of vacuum openings (626) are configured to correspond with the linear alignment of chambers (523) of cassette tray (510) when received within inner chamber (652). In other words, since rotatable member (620) is generally rotatable to place a particular sample chamber (523) of cassette tray (510) into communication with the cutter of needle (22), access openings (622) are oriented on circular base (621) in an arced or semi-circle configuration relative to base manifold (640). However, since cassette tray (510) is generally of a flat configuration, vacuum openings (626) are generally aligned along a common axis. Since each vacuum opening (626) is generally associated with a corresponding access opening (622), only a single vacuum opening (626) is in communication with a vacuum source when the particular corresponding access opening (622) is in communication with the cutter of biopsy device (10). As will also be described in greater detail below, this configuration generally promotes the flow of vacuum into a given vacuum opening (626), into inner chamber (652) (and through cassette tray (510)) and out of a corresponding access opening (622).

As best seen in FIG. 17, within inner chamber (652), lower wall (642) includes a plurality of vacuum walls (641) that define a plurality of vacuum chambers (643). Each vacuum wall (641) extends upwardly from lower wall (642) partially into inner chamber (652). This upward extension both defines vacuum chambers (643) and provides support for cassette tray (510) when cassette tray (510) is inserted into manifold (640). As will be described in greater detail below, each vacuum chamber (643) is in communication with a corresponding vacuum opening (626) to communicate vacuum from probe assembly (20) and into cassette tray (510), as best seen in FIG. 19.

FIG. 20 shows a lower opening (636) of sensor manifold (630) opposite of lower wall (642) of manifold (640). Lower opening (636) is sized and shaped to accommodate a cable or electrical conduit (682) of sensor (680) extending outwardly from sensor manifold (630) when sensor (680) is received therein. As discussed above, lower opening (636) further includes flexible detent (634) extending laterally into lower opening (636). As best seen in FIG. 21, flexible detent (634) is configured to releasably grasp cable (682) of sensor (680) to thereby removably fasten sensor (680) to sensor manifold (630). As will be described in greater detail below, with sensor (680) securely attached to sensor manifold (630), rotatable member (620) is rotated relative to probe assembly (20) to align access openings (622) with the cutter of biopsy device (10) to provide tissue sample holder assembly (600) access to the biopsy site while simultaneously providing immediate imaging of any tissue samples deposited within tissue sample holder assembly (600).

As shown in FIGS. 22-24, an exemplary imaging system (670) comprises a sensor (680) and an imaging device (690). In the present example, sensor (680) is generally configured as a digital sensor, but in other examples sensor (680) can include any other suitable sensor. For instance, in some examples sensor (680) comprises a charge-coupled device (CCD) sensor, a complementary metal-oxide semiconductor (CMOS) sensor, indium gallium arsenide sensors, conventional film, and/or any other sensor as will be apparent to those of ordinary skill in the art. Particularly, as shown in FIG. 23, sensor (680) includes an electronic circuit (683), an imager (684), a fiber optic plate (685) and a scintillator (686) encapsulated within an outer casing (687). Sensor (686) is a diagnostic imaging sensor that is operable to convert and transmit data digitally.

Although not shown, it should be understood that sensor (680) may include additional or alternative internal components than those depicted. For example, sensor (680) may include components corresponding to those included in an interline transfer CCD sensor, frame transfer CCD sensor, on-chip A/D conversion CMOS sensor, off-chip A/D conversion CMOS sensor, those used in short-wave infrared (SWIR) imaging, or thermal imaging. Such internal components of sensor (680) may include various transistors, pixels (photodiodes or photocapacitors), and/or other components as will be apparent to those of ordinary skill in the art. The size and shape of the pixels in sensor (680) may vary to optimize, among other things, the imaging optics, saturation capacities, and signal-to-noise ratios, resolution, spatial frequencies and contrast. The overall size of sensor (680) may also vary to optimize the system's field of view. By way of example only, sensor (680) may be sized as ¼″, ⅓″, ½″, 1/1.8″, ⅔″, 1″, 1.2″ or any other size as will be apparent to those of ordinary skill in the art.

As seen in FIG. 24, imaging device (690) includes a head (692), a base (694) and an extension arm (696) extending therebetween. Extension arm (696) is configured to extend and pivot about base (694) to thereby allow for the selective positioning of head (692). Imaging device (690) is operable to communicate with sensor (680) by transmitting an energy beam (e.g., x-ray, etc.) through air until encountering sensor (680), as seen in FIG. 25. In particular, head (692) is configured to transmit energy beams outwardly upon actuation of imaging device (690). As will be described in greater detail below, any intermediate objects positioned between head (692) and sensor (680), i.e. a biopsied tissue sample, will interact with the energy rays transmitted by imaging device (690) and be identified and depicted in a corresponding image generated by imaging system (670). By way of example only, imaging system (670) may be operable to generate x-ray images (e.g., radiography images), optical coherence tomography images, multipicture or videos, high definition ultrasound images, or other images as will be apparent to those of ordinary skill in the art in view of the teachings herein. Although imaging device (690) of the present example is shown as a boom x-ray source, it should be understood that in other examples, imaging device (690) can take on a variety of forms. For instance, in some examples imaging device (690) can be an x-ray source integrated into a stereotactic imaging system. Thus, it should be understood that in some examples imaging device (690) can be multi-use for both obtaining images of collected samples and for obtaining stereotactic images for targeting purposes.

Although not shown in FIG. 25, it should be understood that in some examples imaging device (690) and/or sensor (680) can be readily integrated into a control module used with biopsy device (10). For instance, in some uses computer image and processing components can be connected to otherwise incorporated into the control module. The control module can then detect when biopsy device (10) has received a tissue sample. Once a tissue sample is received within biopsy device, the control module can then automatically signal imaging device (690) and/or sensor (680) to take an x-ray image of the collected tissue sample.

FIGS. 26-29 show an exemplary use of tissue sample holder assembly (600) to collect tissue samples within cassette tray (510). As best seen in FIG. 26A, with tissue sample holder assembly (600) coupled to probe assembly (20) via coupler (610) in lieu of tissue sample holder assembly (40), cassette tray (510) is then inserted into manifold (640) of rotatable member (620). However, it should be understood that in other uses, cassette tray (510) may be first inserted into manifold (640) and then tissue sample holder assembly (600) may be attached to probe assembly (20). Regardless of whether tissue sample holder assembly (600) is attached to probe assembly (20), cassette tray (510) may be inserted into manifold (640) by inserting distal wall (512) of cassette tray (510) through the proximal end of manifold (640) and into inner chamber (652). Although not shown, it should be understood that in some examples either manifold (640) or cassette tray (510) can include additional sealing features such as rubber gaskets to aid in the sealing of cassette tray (510) relative to the exterior of manifold (640). In other examples, sealing is provided by a compression fit between walls (642, 644, 646) of manifold (640) and an exterior of cassette tray (510).

As seen in FIG. 26B, once cassette tray (510) is inserted into manifold (640), sensor (680) is inserted into sensor manifold (630) of rotatable member (620). As similarly described above, it should be understood that in other uses, sensor (680) may be first inserted into sensor manifold (630) and then tissue sample holder assembly (600) may be attached to probe assembly (20). Alternatively, it should be understood that sensor (680) may be inserted into sensor manifold (630) prior to the insertion of cassette tray (510) into manifold (640) of rotatable member (62). Regardless, rotatable member (62) may receive both cassette tray (510) and sensor (680) prior to the commencement of a medical procedure. In other instances, sensor (680) may be attached to rotatable member (620) after the medical procedure has commenced and at least one tissue sample has been extracted by the cutter of biopsy device (10) and deposited within cassette tray (510). At this stage, after a tissue sample has been communicated into a respective chamber (523) of cassette tray (510), positioned within inner chamber (652) of manifold (640), imaging device (690) is selectively maneuvered towards tissue sample holder assembly (600). In particular, as shown in FIG. 27, with sensor (680) engaged with rotatable member (620) and at least one tissue sample contained within cassette tray (510), head (692) is directed over rotatable member (620) such that manifold (640) is positioned between head (692) and sensor (680).

In this instance, as similarly described above with respect to FIG. 25, imaging system (670) is activated such that imaging device (690) communicates with sensor (680) by transmitting an energy beam (e.g., x-ray, etc.) through air until encountering sensor (680). In particular, head (692) transmits energy beams outwardly towards manifold (640) such that the biopsied tissue samples contained within inner chamber (652) interact with the energy rays transmitted by imaging device (690). Imaging system (670) thereby generates a corresponding image of the tissue samples for examination and analysis by an operator. In other words, the tissue sample absorbs some of the energy or radiation transmitted by head (692), and a corresponding image is generated by imaging system (670) processing the rays received by sensor (680). By way of example only, imaging system (670) may be operable to generate x-ray images (e.g., radiography images), optical coherence tomography images, multipicture or videos, high definition ultrasound images, or other images as will be apparent to those of ordinary skill in the art in view of the teachings herein.

As a result, an image of the tissue sample is immediately generated by imaging system (670) for the benefit of an operator's timely review and assessment. In the present example, an operator is not required to initially disassemble tissue sample holder (600) from biopsy device (10), to subsequently remove the tissue sample from cassette tray (510) for subsequent placement into an examination container (not shown) before being able to generate an image of the tissue sample. Once a first image is taken of the initial tissue sample deposited within manifold (640), tissue sample holder assembly (600) may be moved to an alternative position to fill another chamber (523) of cassette tray (510) with a subsequent tissue sample. In particular, as seen in FIG. 28A, rotatable member (620) may be rotated to a different orientation than that initially shown in FIG. 27 such that a different access opening (622) is aligned with the cutter of biopsy device (10). In particular, rotatable member (620) is rotated relative to coupler (610) and probe assembly (20) by grasping manifold (640) and/or sensor manifold (630) and applying a predetermined force upon rotatable member (620) to thereby rotate of manifold (640), cassette tray (510) contained therein, sensor manifold (630), and sensor (680) contained therein. As a result, rotatable member (620) realigns relative to probe assembly (20) until a subsequent access opening (622) establishes fluid communication with probe assembly (20).

In this instance, a different chamber (523) of cassette tray (510) becomes aligned with the cutter of biopsy device (10). Thus, any subsequent tissue sample(s) extracted by the cutter of biopsy device (10) will be transferred to this subsequent chamber (523) that is now in alignment with the particular access opening (622) that is in communication with probe assembly (20). At this stage, a subsequent image may be taken with the use of imaging system (670) as similarly described above to thereby generate a new image of cassette tray (510) that includes the recently added tissue sample(s) contained in cassette tray (510). It should be understood that the first tissue sample remains within cassette tray (510) when the subsequent image is generated by imaging system (670), such that both tissue samples will appear in the new image taken with imaging device (690). However, due to the isolated arrangement provided in cassette tray (510) by the presence of chambers (523), the tissue samples contained therein will be readily distinguishable such that an operator may easily inspect the multiple tissue samples in the image generated by imaging system (670).

Alternatively, or subsequently, as seen in FIG. 28B, an operator may apply a force upon rotatable member (620) to realign manifold (640) and sensor manifold (630) relative to probe assembly (20) to thereby rotate cassette tray (510) and sensor (680), contained therein, respectively, until an alternate access opening (622) couples with the cutter of biopsy device (10). In this instance, the new chamber (523) of cassette tray (510) that is aligned with the particular access opening (622) that is coupled with probe assembly (20) is now able to receive tissue samples from the cutter of biopsy device (10). After a tissue sample is extracted by the cutter of biopsy device (10) and deposited within a respective chamber (523) of cassette tray (510), an operator may selectively maneuver imaging device (690) as described above to thereby generate an image of the tissue sample. It should be understood that rotatable member (620) may continue to be rotated within coupler (610) to realign a subsequent access opening (622) with the cutter of biopsy device (10) until the desired number of tissue samples have been deposited within cassette tray (510) and imaged by imaging system (670).

Although not shown, it should be understood that rotatable member (620) may be rotated relative to probe assembly (20) in any other order than that depicted and described above. It will also be apparent to those of ordinary skill in the art that an operator may cease rotating rotatable member (620) relative to probe assembly (20) once a sufficient number of tissue samples have been deposited within cassette tray (510). In other words, although tissue sample holder assembly (600) is described above as being used to collect a tissue sample in each chamber (523) of cassette tray (510), it should be understood that in some uses it may be desirable to only collect samples in one or more specific chambers (523) of cassette tray (510). Accordingly, in some uses rotatable member (620) may be rotated to skip some chambers (523). Similarly, it should be understood that an operator is not required to further rotate rotatable member (620) through to each access opening (622) iteration. Rather, an operator may simply cease rotation rotating member (620) when the procedure is completed. It should also be understood that cassette tray (510) may be removed from manifold (622) at any point during the engagement of tissue sample holder assembly (600).

FIG. 29 shows a path (80) of a tissue sample (90) traveling through probe assembly (20) towards coupler (610) until arriving at the particular access opening (622) that is currently aligned with the cutter of biopsy device (10). In this instance, with a particular access opening (622) of manifold (640) aligned with the cutter of biopsy device (10), depending on the rotatable orientation of rotatable member (620) relative to probe assembly (20), tissue sample (90) enters manifold (640) and is directed through the channel of access opening (622) until encountering cassette tray (510). For the access openings (622) aligned with raised portion (645), it should be understood that when tissue sample (90) is received within inner chamber (652) tissue sample (90) is directed downward toward cassette tray (510) from the downward angle of raised portion (645). Thus, it should be understood that the angle of raised portion (645) acts as a tissue sample deflector or director to direct tissue sample (90) into cassette tray (510) after tissue sample (90) is received through the respective access openings (622) that are positioned in alignment with raised portion (645). Accordingly, although raised portion (645) is shown as having a specific angle and/or geometry, it should be understood that the particular configuration of raised portion (645) may vary based on a number of considerations such as the positioning of access openings (622) relative to manifold (640), the velocity of tissue sample (90) transported from probe assembly (20), the size of tissue samples (90), the gage size of needle (22), and/or etc.

With tissue sample (90) deposited within a particular chamber (523) of cassette tray (540), chamber (523) isolates tissue sample (90) from any other tissue samples (90) previously deposited, or to be deposited, within other chambers (523) of cassette tray (510). Although not shown, it should be understood that a seal may be included within inner chamber (652) of manifold (640) that is configured to form an airtight seal against cassette tray (510) when received within manifold (640). In this instance, the seal of manifold (640) will abut a proximal portion of cassette tray (510) to prevent any fluid and/or tissue samples (90) from exiting cassette trays (510).

With cassette tray (510) positioned within inner chamber (652), vacuum walls (641) extending upwardly from lower wall (622) maintain cassette tray (510) at an elevated position to thereby provide ample space for vacuum chambers (643) to receive any fluid dispensed from tissue samples (90) deposited therein. In other words, vacuum walls (641) extend along lower wall (622) in parallel alignment with chambers (523) of cassette tray (510) when inserted into inner chamber (652). Vacuum walls (641) extend along the substantial longitudinal length of cassette tray (510) to thereby provide support for cassette tray (510) when received within manifold (640). Vacuum chambers (643) similarly extends along lower wall (622) and are in fluid communication with cassette tray (510) through vents (526) of floor (524). Vacuum chambers (643) are in communication with a respective vacuum opening (626) to communicate vacuum from probe assembly (20) into cassette tray (510). In particular, vacuum enters manifold (640) through the particular vacuum opening (626) that is in communication with a vacuum port (76) of probe assembly (20). Next, vacuum travels through vacuum chamber (626) and upwardly through vents (526) of cassette tray (510). Vacuum then travels through into inner chamber (652) of manifold (640). In this instance, the vacuum is now in communication with the particular access opening (622) that is currently aligned with the cutter of biopsy device (10). Vacuum is then used to pull tissue sample (90) through needle (22) and into the corresponding chamber (523) of cassette tray (510).

Once a sample is received within chamber (523) of cassette tray (510), rotatable member (620) is rotated to a subsequent position relative to probe assembly (20), as shown in FIGS. 28A-28B. This translation indexes the next successive access opening (622) and chamber (523) of cassette tray (510) to receive tissue sample (90) therein by being in communication with the cutter of biopsy device (10) as similarly described above. In this position, another tissue sample (90) may be collected in cassette tray (510), corresponding to the selected access opening (622) of manifold (640), and a subsequent image may be generated by of tissue sample (90) by imaging system (670). Once the desired number of images of tissue samples (90) contained in cassette tray (510) are taken, cassette tray (510) is removed from manifold (640) by pulling proximal end (516) proximally to thereby extract floor (524) from within inner chamber (652).

With cassette tray (510) completely removed from rotatable member (620), an operator may next desire to perform further analysis on the collected tissue samples (90). At this stage, cassette tray (510) may be manipulated for a visual inspection of each tissue sample to supplement the visual inspection of the generated images of the tissue samples. If an operator is not satisfied with the results at this stage, undesirable tissue samples may be discarded and the same cassette tray (510) may be inserted back into manifold (640) of rotatable member (620) for collection of addition tissue samples. Alternatively, an entirely new cassette tray (510) may be placed into manifold (640) of rotatable member (620) for collection of additional tissue samples.

Once tissue sample are collected to the satisfaction of an operator, the operator may desire to transport tissue samples to a pathology laboratory. At this stage, an operator may mark or place a label onto labeling portion (528) to ensure chain of custody through the workflow. Alternatively, in some uses, labeling portion (528) may already be labeled at this stage. For instance, in some uses labeling potion (528) may be labeled at the beginning of the biopsy procedure before collecting any tissue samples. Alternatively, in some uses labeling portion (528) may be prelabeled with a bar code, QR code, or another computer readable medium. Where such computer readable mediums are used, labeling portion (528) may be scanned as various stages to associate the computer readable medium with the patient. This may include multiple scans throughout the procedure such as before the biopsy procedure, after collection of tissue samples, after procedure room x-ray, and/or etc.

Once chain of custody has been established using labeling portion (528), cassette tray (510) may be inserted into cover (540) as described above. The combination of cassette tray (510) and cover (540) may then be inserted into jar (160) described above. As described above, jar (160) may be filled with a fluid such as formalin to preserve the collected tissue samples during transport and/or storage. Although cassette tray (510) is described herein as being used with the same jar (160) described above, it should be understood that other alternative jars or containers may be used for transport and/or storage of cassette tray (510). For instance, in some examples jar (160) may be replaced with a container of a variety of shapes and sizes. In other examples, cover (540) itself may be used to transport cassette tray (510). Of course, in such examples structures of cover (540) such as vents (544) and/or open spaces (549) can be closed so that cover (540) can hold fluids such as formalin.

After the combination of cassette tray (510) and cover (540) is inserted into jar (160), jar (160) may be transported to the pathology laboratory as shown in FIG. 7 and described above. The collected tissue samples may then be processed in accordance with the workflow (300) shown in FIG. 7. However, since cassette assembly (500) can be used in lieu of tissue processing cassette (200), it should be understood that certain steps may be omitted such as straining the collected samples as represented by box (350) and placing the collected samples into a tissue processing cassette (200) as represented by box (370). In addition, it should be understood that at any one or more of the steps depicted in FIG. 7, an operator may interact with labeling portion (528) to confirm chain of custody of the collected tissue samples. By way of example only, this may include scanning computer readable mediums associated with labeling portion (528), confirming information on labeling portion (528) with information on jar (160) or other components, or confirming information on labeling portion (528) with patient files.

IV. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### EXAMPLE 1

A biopsy device, comprising: (a) a body; (b) a needle extending distally from the body; (c) a tissue sample holder, wherein the needle is in communication with the tissue sample holder, wherein the tissue sample holder includes a rotatable member defining a cassette housing and a sensor housing; (d) a cassette tray including a plurality of tissue sample chambers, wherein the cassette tray is configured for receipt within the cassette housing of the tissue sample holder; and (e) a sensor, wherein the sensor is operable to digitally convert and transmit data, wherein the sensor is configured for receipt within the sensor housing of the tissue sample holder; wherein the rotatable member is rotatable relative to the body.

### EXAMPLE 2

The biopsy device of Example 1, further comprising an imaging device operable to communicate with the sensor.

### EXAMPLE 3

The biopsy device of Example 2, wherein the imaging device is configured to transmit energy beams towards the tissue sample holder.

### EXAMPLE 4

The biopsy device of any one or more of Examples 2 through 3, wherein the imaging device and the sensor are configured to cooperate to generate x-ray images of tissue samples received within the cassette tray.

### EXAMPLE 5

The biopsy device of any one or more of Examples 1 through 4, wherein the sensor includes an electronic circuit, an imager, a fiber optic plate, and a scintillator.

### EXAMPLE 6

The biopsy device of any one or more of Examples 1 through 5, wherein the sensor housing is positioned adjacent to the cassette housing.

### EXAMPLE 7

The biopsy device of any one or more of Examples 1 through 6, wherein the sensor is configured to digitally transmit data in response to the energy from the imaging device.

### EXAMPLE 8

The biopsy device of any one or more of Examples 2 through 5, wherein the imaging device comprises a beam transmitter.

### EXAMPLE 9

The biopsy device of Example 8, wherein the beam transmitter is a boom-mounted x-ray source.

### EXAMPLE 10

The biopsy device of Example 8, wherein the beam transmitter is a handheld x-ray source.

### EXAMPLE 11

The biopsy device of any one or more of Examples 1 through 10, wherein the cassette tray is removably attached to the cassette housing by a seal such that the cassette tray is removable from the tissue sample holder.

### EXAMPLE 12

The biopsy device of Example 11, wherein the seal is configured to generate a compression fit against the cassette tray when received in the cassette housing.

### EXAMPLE 13

The biopsy device of any one or more of Examples 1 through 13, wherein the sensor is removably attached to the sensor housing by a flexible detent such that the cassette tray is removable from the tissue sample holder.

### EXAMPLE 14

The biopsy device of any one or more of Examples 1 through 13, wherein the tissue sample holder includes a coupler configured to releasably couple the cassette housing and sensor housing to the body.

### EXAMPLE 15

The biopsy device of any one or more of Examples 1 through 14, wherein the cassette housing is configured to receive at least one tissue sample from the needle such that the at least one tissue sample is deposited within the cassette tray contained within the cassette housing.

### EXAMPLE 16

The biopsy device of any one or more of Examples 1 through 15, wherein the cassette housing includes at least one access opening configured to receive tissue samples from the body.

### EXAMPLE 17

The biopsy device of Example 16, wherein the cassette housing includes four access openings.

### EXAMPLE 18

The biopsy device of any one or more of Examples 16 through 17, wherein the cassette housing is configured to selectively rotate between the four access openings to thereby provide fluid communication between each access opening of the four access opening and the needle.

### EXAMPLE 19

The biopsy device of any one or more of Examples 1 through 19, wherein the cassette housing includes at least one vacuum opening configured to provide a vacuum within the cassette housing.

### EXAMPLE 20

The biopsy device of any one or more of Examples 16 through 19, wherein the cassette housing includes four vacuum openings in association with the four access openings.

### EXAMPLE 21

The biopsy device of any one or more of Examples 19 through 20, wherein the cassette housing includes at least one channel extending from the at least one vacuum opening.

### EXAMPLE 22

The biopsy device of any or more of Examples 19 through 21, wherein the cassette housing is configured to selectively rotate between the four vacuum openings to thereby provide fluid communication between a particular vacuum opening of the four vacuum openings and the body.

### EXAMPLE 23

The biopsy device of any one or more of Examples 1 through 22, wherein the cassette tray further includes one or more indicia, wherein each indicia of the one or more indicia corresponds to a respective sample chamber.

### EXAMPLE 24

The biopsy device of Example 23, wherein the plurality of chambers of the cassette tray includes a proximal wall, wherein the one or more indicia are disposed on the proximal wall.

### EXAMPLE 25

The biopsy device of any one or more of Examples 23 through 24, wherein the one or more indicia are configured to be visible under x-ray imaging.

### EXAMPLE 26

A method of taking an image of a biopsied tissue sample from a biopsy device, the biopsy device including a body, a needle, and a tissue sample holder, the method comprising the steps of: (a) inserting the needle into tissue to extract a sample; (b) inserting a cassette tray into the tissue sample holder, wherein the cassette tray includes a plurality of chambers; (c) rotating the tissue sample holder relative to the body to align one of the plurality of chambers with the needle; (d) transferring the tissue sample from the needle to the tissue sample holder such that the tissue sample is stored in the chamber of the cassette tray; (e) inserting a sensor into the tissue sample holder such that the sensor is positioned adjacent to the cassette tray; (f) positioning an imaging device adjacent to the tissue sample holder such that the imaging device is aligned toward the cassette tray and the sensor; and (e) activating the imaging device to transmit energy toward the tissue sample and the sensor.

### EXAMPLE 27

The method of Example 26, further comprising generating an image of the tissue sample.

### EXAMPLE 28

The method of Example 27, further comprising displaying a graphical representation or depiction of the tissue sample.

### EXAMPLE 29

The method of any one or more of Examples 26 through 28, the method further comprising rotating the tissue sample holder to align a different chamber of the plurality of chambers with the needle.

### EXAMPLE 30

A biopsy device, comprising: (a) a body; (b) a cutter extending distally from the body; and (c) a tissue sample holder, wherein the cutter is in communication with the tissue sample holder, wherein the tissue sample holder is selectively rotatable relative to the body, wherein the tissue sample holder includes a first port and a second port; wherein the first port is configured to slidably receive a cassette tray therein, wherein the second port is configured to slidably receive a sensor configured to digitally convert and transmit data therein.

### EXAMPLE 31

A biopsy device, comprising: (a) a body; (b) a needle extending distally from the body; and (c) a tissue sample holder, wherein the needle is in communication with the tissue sample holder, wherein the tissue sample holder includes a rotatable member defining a at least one housing, wherein the at least one housing is configured to releasably hold a cassette tray and sensor therein, wherein the rotatable member is configured to rotate relative to the body to position the cassette tray in a plurality of tissue collection positions.

### EXAMPLE 32

The biopsy device of Example 31, wherein the tissue sample holder includes a second housing configured to releasably hold the sensor therein.

### EXAMPLE 33

The biopsy device of Example 32, wherein second housing is configured to position the sensor relative to the cassette tray to permit collection of data via the sensor related to one or more tissue samples collected within the cassette tray.

### EXAMPLE 34

A biopsy device, comprising: (a) a body; (b) a needle extending distally from the body; and (c) a tissue sample holder, wherein the needle is in communication with the tissue sample holder, wherein the tissue sample holder includes a rotatable member defining a at least one housing, wherein the at least one housing is configured to releasably hold a sensor therein, wherein the rotatable member is configured to rotate relative to the body.

### EXAMPLE 35

A biopsy device, comprising: (a) a body; (b) a needle extending distally from the body; and (c) a tissue sample holder, wherein the needle is in communication with the tissue sample holder, wherein the tissue sample holder includes a rotatable member defining a first housing and a second housing, wherein the first housing is sized and shaped to receive a tray, wherein the tray includes a plurality of dividers, wherein the tray is configured to releasably detach from within the first housing of the tissue sample holder; wherein the second housing is sized and shaped to receive a data transmitter, wherein the data transmitter is configured to releasably detach from within the second housing of the tissue sample holder; wherein the rotatable member is configured to rotate relative to the body such that the tray and the data transmitter are operable to rotate with the rotatable member when received with the first and second housings.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

It should be understood that any of the versions of instruments described herein may include various other features in addition to or in lieu of those described above. By way of example only, any of the instruments described herein may also include one or more of the various features disclosed in any of the various references that are incorporated by reference herein. It should also be understood that the teachings herein may be readily applied to any of the instruments described in any of the other references cited herein, such that the teachings herein may be readily combined with the teachings of any of the references cited herein in numerous ways. Other types of instruments into which the teachings herein may be incorporated will be apparent to those of ordinary skill in the art.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

We claim:

1. A biopsy device, comprising:
(a) a body;
(b) a needle extending distally from the body;
(c) a tissue sample holder, wherein the needle is in communication with the tissue sample holder, wherein the tissue sample holder includes a rotatable member defining a cassette mount and a sensor mount proximate to the cassette mount;
(d) a tissue processing cassette sized to be used in a pathology laboratory for purposes of dehydrating, embedding and sectioning, wherein the tissue processing cassette is configured for receipt within the cassette mount of the tissue sample holder for receipt of one or more tissue samples therein; and
(e) an x-ray sensor configured to be received within the sensor mount and adapted to receive an x-ray that has passed through one or more tissue samples received in the tissue processing cassette.

2. The biopsy device of claim 1, further comprising an imaging device configured to communicate with the x-ray sensor.

3. The biopsy device of claim 1, further comprising an imaging device configured to communicate with the x-ray sensor, wherein the imaging device is movable relative to the tissue sample holder.

4. The biopsy device of claim 1, further comprising an imaging device configured to communicate with the x-ray sensor, wherein the imaging device and the x-ray sensor are configured to cooperate to generate x-ray images of tissue samples received within the tissue processing cassette.

5. The biopsy device of claim 1, wherein the sensor mount is positioned adjacent to the cassette mount.

6. The biopsy device of claim 1, further comprising an imaging device configured to communicate with the x-ray sensor, wherein the imaging device is a boom-mounted x-ray source.

7. The biopsy device of claim 1, further comprising an imaging device configured to communicate with the x-ray sensor, wherein the imaging device is a handheld x-ray source.

8. The biopsy device of claim 1, wherein the x-ray sensor is removably attached to the x-ray sensor mount by a flexible detent such that the x-ray sensor is selectively removable from the tissue sample holder.

9. The biopsy device of claim 1, wherein the sensor mount includes a lower opening sized to accommodate a cable extending from the x-ray sensor.

10. The biopsy device of claim 1, wherein the cassette mount includes a plurality of vacuum openings and a plurality of sample openings, wherein each vacuum opening is associated with a corresponding sample opening.

11. The biopsy device of claim 1, wherein the cassette mount includes a plurality of vacuum openings and a plurality of sample openings, wherein each vacuum opening is associated with a corresponding sample opening, wherein each sample opening is laterally offset relative to an adjacent sample opening such that the sample openings together are positioned in an arcuate configuration.

12. The biopsy device of claim 1, further comprising a control module, wherein the control module is in communication with the x-ray sensor to automatically collect an x-ray image after receipt of a tissue sample within the tissue processing cassette.

13. The biopsy device of claim 1, further comprising an imaging device configured to communicate with the x-ray sensor, wherein the imaging device and the x-ray sensor are configured to cooperate to generate x-ray images of tissue samples received within the tissue processing cassette, wherein the imaging device is further operable to generate x-ray images of a patient in cooperation with patient x-ray sensor.

14. The biopsy device of claim 1, wherein the cassette mount defines a cassette chamber and a vacuum chamber, wherein the vacuum chamber is positioned below the cassette chamber and above the sensor mount.

15. The biopsy device of claim 1, wherein the cassette mount defines a cassette chamber and a vacuum chamber, wherein the vacuum chamber is positioned below the cassette chamber and above the sensor mount, wherein the vacuum chamber includes a plurality of walls to direct vacuum into different tissue sample chambers defined by the tissue processing cassette.

16. A tissue sample holder for use with a biopsy device, the tissue sample holder comprising:
(a) a rotatable member defining a cassette mount and a sensor mount laterally offset relative to the cassette mount;
(d) a tissue processing cassette sized to be used in a pathology laboratory for purposes of dehydrating, embedding and sectioning, wherein the tissue processing cassette is configured for receipt within the cassette mount for receipt of one or more tissue samples therein; and
(e) an x-ray sensor configured to be received within the sensor mount and adapted to receive an x-ray that has passed through one or more tissue samples received in the tissue processing cassette.

17. The tissue sample holder of claim 16, wherein the cassette mount includes plurality of sample openings and a raised portion in communication with one or more of the sample openings, wherein the raised portion is configured to deflect tissue samples into the tissue processing cassette.

18. The tissue sample holder of claim 16, wherein the cassette mount includes plurality of sample openings, a plurality of vacuum passages, and a raised portion in communication with one or more of the sample openings, wherein the raised portion is configured to deflect tissue samples into the tissue processing cassette under vacuum supplied by one or more of the vacuum passages corresponding to the one or more sample openings in communication with the raised portion.

19. The tissue sample holder of claim 16, wherein the cassette mount includes plurality of sample openings, a plurality of vacuum passages, wherein each sample opening is associated with a corresponding vacuum passage, wherein the rotatable member is rotatable relative to the biopsy device to index each sample opening and each corresponding vacuum passage with the biopsy device.

20. A method of taking an image of a biopsied tissue sample from a biopsy device, the biopsy device including a body, a needle, and a tissue sample holder, the method comprising the steps of:
(a) inserting a tissue processing cassette into the tissue sample holder, wherein the tissue processing cassette is sized to be used in a pathology laboratory for purposes of dehydrating, embedding and sectioning, wherein the tissue processing cassette defines a plurality of tissue chambers;
(b) rotating the tissue sample holder relative to the body to align the selected one tissue chamber of the plurality of tissue chambers with the needle;
(c) cutting a tissue sample using the needle and transporting the tissue sample to the tissue sample holder such that the tissue sample is disposed in the selected one tissue chamber of the tissue processing cassette;
(d) inserting a sensor into the tissue sample holder such that the sensor is positioned proximate the tissue processing cassette;
(e) activating an imaging device positioned towards the sensor to thereby capture an x-ray image of the selected one tissue sample using the sensor.
